(19) United States
(12) Patent Application Publication
(10) Pub. No.: US 2022/0301672 A1
(43) Pub. Date: Sep. 22, 2022

(54) COMPUTING DEVICE WITH IMPROVED USER INTERFACE FOR INTERPRETING AND VISUALIZING DATA

(71) Applicant: Roche Sequencing Solutions, Inc., Pleasanton, CA (US)

(72) Inventors: Anoop Grewal, Cupertino, CA (US); Shuba Krishna, Los Altos, CA (US); Marco Maetzler, Belmont, CA (US); Matteus Jiawei Pan, San Francisco, CA (US); Maya Venkatraman, Palo Alto, CA (US)

(21) Appl. No.: 17/805,511

(22) Filed: Jun. 6, 2022

Related U.S. Application Data
(63) Continuation of application No. 16/022,835, filed on Jun. 29, 2018, now abandoned.

(51) Int. Cl. G16H 15/00; G16H 10/60; G16H 20/10; G16B 45/00; G16B 50/00

(52) U.S. Cl. CPC G16H 15/00; G16H 10/60; G16H 20/10; G16B 45/00; G16B 50/00

(57) ABSTRACT

The present disclosure provides, in some embodiments, a computing device comprising an improved user interface. In some embodiments, the improved user interface enables the visualization of clinically relevant information pertaining to interacting gene variants, including therapeutic recommendations. In some embodiments, the improved user interface facilitates the contemporaneous visualization of clinically relevant information pertaining to individual gene variants and the visualization of clinically relevant information pertaining to an interaction between gene variants, including therapeutic recommendations. In some embodiments, the visualization(s), through the improved user interface, facilitates the rapid interpretation of clinically relevant information by a medical professional such that decisions regarding patient care may be made accurately and efficiently.

### CROSS REFERENCE TO RELATED APPLICATIONS

This application is a continuation of U.S. patent application Ser. No. 16/022,835, filed on Jun. 29, 2018, the disclosure of which is hereby incorporated by reference herein in its entirety.

### BACKGROUND OF THE DISCLOSURE

Studies of human genetic variation using DNA sequencing have undergone an extraordinary development from their introduction over 40 years ago up to current technologies, which allow for a human genome to be sequenced and analyzed within a matter of days. The release of the first “next-generation sequencing” (NGS) instruments in the mid-2000s led to a revolution in disease study, offering vastly improved speed at significantly lower cost—enabling the generation of a whole human genome sequence in a matter of weeks. In addition to price and performance, the new sequencing technology also proved to compensate for some of the technical weaknesses of the older sequencing and genotyping technologies, allowing for the genome-wide detection of variants, including novel ones, at a low cost. A further breakthrough for NGS in human genomics arrived with the introduction of targeted enrichment methods, allowing for selective sequencing of regions of interest, thereby dramatically reducing the amount of sequences that needed to be generated. The approach is based on a collecting DNA or RNA probes representing the target sequences in the genome, which can bind and extract the DNA fragments originating from targeted regions.

Whole exome sequencing (WES), which enables sequencing of all protein-coding regions in the human genome (the exome) quickly became the most widely used targeted enrichment method, especially for monogenic (“Mendelian”) diseases. This approach enabled the detection of both exonic (coding) as well as splice-site variants, while requiring only approximately 2% of sequencing “load” compared to whole genome sequencing. The unbiased analysis of all genes eliminated the need for a time-consuming selection of candidate genes prior to sequencing. It has been estimated that the exome harbors about 85% of mutations with large effects on disease-related traits. In addition, exonic mutations were shown to cause the majority of monogenic diseases, with missense and nonsense mutations alone accounting for approximately 60% of disease mutations.

Recent advances in genome sequencing technologies provide unprecedented opportunities to characterize individual genomic landscapes and identify mutations relevant for diagnosis and therapy. Indeed, in recent years, NGS has also been increasingly applied for addressing pharmacogenomic research questions. It is not only possible to detect genetic causes that explain why some patients do not respond to a certain drug, but also try to predict a drug's success based on genetic information. Certain genetic variants can affect the activity of a particular protein and these can be used to estimate the probable efficacy and toxicity of a drug targeting such a protein. NGS therefore has applications far beyond finding disease-causing variants.

About 99.5% of all DNA is shared across all humans; it is the 0.5% that makes all the difference. Genetic variations, or variants, are the differences that make each person's genome unique. DNA sequencing identifies an individual's variants by comparing the DNA sequence of an individual to the DNA sequence of a reference genome maintained by the Genome Reference Consortium (GRC). It is believed that the average human's genome has millions of variants. Some variants occur in genes but most occur in DNA sequences outside of genes. A small number of variants have been linked with diseases, but most variants have unknown effects. Some variants contribute to the differences between humans, such as different eye colors and blood types. As more DNA sequence information becomes available to the research community, the effects of some variants may be better understood.

Understanding the effects of variants is a complicated process. While there are many publicly available sources of information about variants and their classification based on American College of Medical Genetics and Genomics guidelines that link variants to diseases, knowledge about variants is constantly evolving and inconsistencies and inaccuracies can be found in these sources. In addition, co-occurring mutations can alter the response to therapy and it is a challenge for the clinician to analyze all possible permutations of variant combinations and interpret the impact to therapy.

### BRIEF SUMMARY OF THE DISCLOSURE

The present disclosure provides, in some embodiments, a computing device comprising an improved user interface. In some embodiments, the improved user interface enables the visualization of clinically relevant information pertaining to interacting gene variants, including therapeutic recommendations. In some embodiments, the improved user interface facilitates the contemporaneous visualization of clinically relevant information pertaining to individual gene variants and the visualization of clinically relevant information pertaining to an interaction between gene variants, including therapeutic recommendations. It is believed that the visualization(s), through the improved user interface, facilitates the rapid interpretation of clinically relevant information by a medical professional such that decisions regarding patient care may be made accurately and efficiently. It is further believed that the systems and methods disclosed herein provide medical professionals with visualizations that enable quick and efficient interpretation of clinically relevant information.

In some embodiments, the present disclosure also provides a system and method which facilitates the review, interpretation, and visualization of clinically relevant information pertaining to one or more identified gene variants derived from a sequenced nucleic acid sample, such as a nucleic acid sample obtained from a human subject. The present disclosure further provides a system and method which facilitates the review, interpretation, and visualization of clinically relevant information as it pertains to the positive or negative interaction between at least two identified gene variants. It is believed that the use of a curated database, including one that includes information pertaining to the interaction of multiple of gene variants as they relate to a specific disease or condition, could eliminate inconsistencies and inaccuracies while enabling improved access to relevant treatment options for clinicians and their patients. Moreover, the systems described herein are efficient, enabling rapid analysis and identification of variants from obtained sequence data, and the quick retrieval of all relevant curated data associated with the identified one or more variants (including interacting gene variants, such as described herein).

In one aspect of the present disclosure is a computing device (100) comprising a display screen (108), the computing device (100) configured to: obtain a plurality of gene variants from one or more memories (104) communicatively coupled to the computing device (100), the plurality of gene variants having been derived from sequence data derived from a patient sample; display on the display screen (108) a first representation comprising data pertaining to an identified clinically relevant interaction between at least two gene variants of the plurality of gene variants; and contemporaneously with displaying the first representation, display on the display screen (108) a second representation comprising clinically relevant information pertaining to a first of the at least two gene variants for which an interaction was identified. In some embodiments, a third representation comprising clinically relevant information pertaining to a second of the at least two gene variants for which an interaction was identified is displayed on the display screen (108).

In some embodiments, the data pertaining to an identified clinically relevant interaction is a therapeutic recommendation based on the interaction between the at least two gene variants. In some embodiments, therapeutic recommendation is not to administer a particular drug or treatment protocol. In some embodiments, the therapeutic recommendation is a therapy sensitive for a disease of interest. In some embodiments, the therapy sensitive for the disease of interest is marked with a first indicia (see, e.g., FIG. 5C). In some embodiments, a therapy resistant for the disease of interest is marked with a second indicia (see, e.g., FIG. 5C). In some embodiments, the first and second representations are displayed within a single panel (see, e.g., FIG. 5C). In some embodiments, the single panel is an individual gene panel representation (see 530 of FIG. 5A). In some embodiments, the second representation is visualized in a first portion of the individual gene panel representation and wherein the first representation is visualized in a second portion of the individual gene panel representation. In some embodiments, the first and second representations are displayed within separate panels. In some embodiments, the first representation is displayed within an interacting gene panel (see 510 or 520 of FIG. 5A), and wherein the second representation is displayed within an individual gene panel. In some embodiments, the individual gene panel further comprises the first representation and an identification of the first of the at least two gene variants.

In another aspect of the present disclosure is a method of presenting on a display coupled to a computing device relevant information pertaining to the presence of one or more gene mutations in a sample derived from a patient, the method comprising: obtaining, from a memory (or one or more memories) coupled to the computing device, a plurality of gene variants (e.g. gene mutations) within sequence data derived from the patient's sample (e.g. a tissue sample; a tumor tissue sample); accessing one or more databases to identify a clinically relevant interaction between at least two of the obtained gene variants from the plurality of obtained gene variants; and displaying at least one visualization comprising the identified clinically relevant interaction. In some embodiments, the method further comprises generating a report including the at least one visualization.

In some embodiments, the at least one visualization comprising the identified clinically relevant interaction is a panel providing one or more therapeutic recommendations based on the identified clinically relevant interaction. In some embodiments, therapeutic recommendation is not to administer a particular drug or treatment protocol. In some embodiments, each of a series of drugs is identified but “greyed out” within the panel (see, e.g., FIG. 5C).

In some embodiments, the therapeutic recommendation is a therapy sensitive for a disease of interest. In some embodiments, the therapy sensitive for the disease of interest is marked with a first indicia. In some embodiments, a therapy resistant for the disease of interest is marked with a second indicia (see, e.g., FIG. 5D). In some embodiments, the second indicia is text which is “greyed out.” In some embodiments, the first indicia is a solid symbol (e.g. a solid circle); and wherein the second indicia is a hollow symbol (e.g. an open or hollow circle).

In some embodiments, the panel is an interacting gene panel representation. In some embodiments, the interacting gene panel representation includes a first portion listing the at least two obtained gene variants for which the clinically relevant interaction was identified, and a second portion reporting the therapeutic recommendation. In some embodiments, the panel is an individual gene panel representation. In some embodiments, the individual gene panel representation includes a first portion listing one of the at least two obtained gene variants for which the clinically relevant interaction was identified, and a second portion reporting the therapeutic recommendation.

In some embodiments, the identified clinically relevant interaction pertains to a disease of interest. In some embodiments, the disease of interest is a pre-diagnosed disease. In some embodiments, the pre-diagnosed disease is cancer. In some embodiments, the one or more databases are stored on one or more remote servers. In some embodiments, the one or more databases comprise curated clinical information. In some embodiments, at least one of the one or more databases comprises clinical information pertaining to the interaction of gene variants. In some embodiments, a first database is accessed for retrieval of clinically relevant information pertaining to at least a first obtained gene variant from the plurality of obtained gene variants for a disease of interest; and wherein a second database is accessed for retrieval of clinically relevant information pertaining to an interaction between the at least the first obtained gene variant and at least a second obtained gene variant from the plurality of obtained gene variants for the same disease of interest. In some embodiments, accessing the one or more databases comprises accessing a first and a second table stored in the one or more databases, the first table comprising information about the gene variants, and wherein the second table comprises information about gene variant interactions.

In some embodiments, the method further comprises filtering the obtained plurality of gene variants by applying at least one quality metric filter. In some embodiments, at least two visualizations are displayed, wherein one of the at least two visualizations comprises an individual gene panel representation.

In another aspect of the present disclosure is a method of presenting on a display coupled to a computing device clinically relevant for a disease of interest (e.g. a diagnosed disease), the method comprising: (i) obtaining variant data derived from a nucleic acid sample obtained from a patient, where the variant data is obtained from one or more memories communicatively coupled to the computing device; (ii) optionally receiving, on a graphical user interface, a first user input corresponding to one or more user configurable filtering settings such that the obtained variant data may be filtered; (iii) identifying clinically relevant information pertaining to an interaction between at least two of the obtained gene variants by accessing one or more databases comprising clinically relevant information; and (iv) displaying, on the graphical user interface, a visualization comprising the clinically relevant information pertaining to the interaction between the at least two gene variants. In some embodiments, the method further comprises generating a report including at least the visualization comprising the clinically relevant information pertaining to the interaction between the at least two gene variants.

In some embodiments, the one or more user configurable filtering settings include user configurable variant quality settings and user configurable genetic database presence settings. In some embodiments, the user configurable variant quality settings include read depth and variant allele frequency. In some embodiments, the one or more user configurable filtering settings comprise (i) adding annotations; (ii) including or excluding therapeutic options from the identified clinically relevant information; and (iii) reclassifying a tier rating of identified clinically relevant information.

In some embodiments, the visualizations are interacting gene panel representations having a first portion listing the at least two gene variants that are interacting, and a second portion reporting a therapeutic recommendation. In some embodiments, the therapeutic recommendation is a therapy sensitive for the disease of interest in the presence of the gene interaction between the at least two obtained gene variants. In some embodiments, the therapy sensitive for the disease of interest is marked within the second panel with a first indicia. In some embodiments, therapy resistant to the disease of interest in view of the interaction between the at least two obtained gene variants is marked within the second panel with a second indicia.

In some embodiments, the method further comprises displaying, on the graphical user interface, an identification of clinically relevant information pertaining to one or more individual gene variants derived from the filtered variant data. In some embodiments, the method further comprises displaying one or more individual gene panel representations, wherein at least one of the individual gene panel representations includes clinically relevant information pertaining to one of the at least two gene variants for which an interaction was identified. In some embodiments, the individual gene panel representations further include at least one of a variant allele frequency statistic or a read depth statistic.

In another aspect of the present disclosure is a method comprising: displaying, on a computing device having a display screen, a first representation comprising data pertaining to an identified clinically relevant interaction between at least two gene variants of a plurality of gene variants; displaying, on the display screen, a second representation comprising clinically relevant information pertaining to at least one of the at least two gene variants for which an interaction was identified. In some embodiments, the plurality of gene variants are derived from sequence data derived from a patient sample. In some embodiments, the plurality of gene variants are obtained from one or more memories communicatively coupled to the computing device.

In some embodiments, the data pertaining to an identified clinically relevant interaction is a therapeutic recommendation based on the interaction between the at least two gene variants. In some embodiments, the therapeutic recommendation is not to administer a particular drug or treatment protocol. In some embodiments, the therapeutic recommendation is a therapy sensitive for a disease of interest. In some embodiments, the therapy sensitive for the disease of interest is marked with a first indicia; and wherein a therapy resistant for the disease of interest is marked with a second indicia. In some embodiments, the first and second representations are displayed within a single panel, wherein the single panel is an individual gene panel representation. In some embodiments, the second representation is visualized in a first portion of the individual gene panel representation and wherein the first representation is visualized in a second portion of the individual gene panel representation. In some embodiments, the first and second representations are displayed within separate panels.

In another aspect of the present disclosure is a non-transitory computer-readable medium storing instructions which, when executed by one or more processors of a computing system, causes the computing system to display on the display screen a first representation comprising data pertaining to an identified clinically relevant interaction between at least two gene variants of a plurality of gene variants, and to additionally display on the display screen a second representation comprising clinically relevant information pertaining to at least one of the at least two gene variants for which an interaction was identified, wherein the plurality of gene variants are derived from sequence data derived from a patient sample. In some embodiments, the plurality of gene variants are obtained from one or more memories communicatively coupled to the computing. In some embodiments, the data pertaining to an identified clinically relevant interaction is a therapeutic recommendation based on the interaction between the at least two gene variants.

### BRIEF DESCRIPTION OF THE FIGURES

The patent or application file contains at least one drawing executed in color. Copies of this patent or patent application publication with color drawings will be provided to the Office upon request and the payment of the necessary fee.

For a general understanding of the features of the disclosure, reference is made to the drawings. In the drawings, like reference numerals have been used throughout to identify identical elements.

FIG. 1 illustrates a system including a computer having one or more processors and a sequencing device, where the computer and the sequencing device are communicatively coupled, such as through a network, in accordance with some embodiments.

FIG. 2 illustrates a system including a processing subsystem, a storage subsystem, an output device, and an input device, each of the components communicatively coupled through a bus, a network, or other wired or wireless interconnect, in accordance with some embodiments. The system may also include software to enable remote access, i.e. a client portal or client interface.

FIG. 3 sets forth a block diagram of a system communicatively coupled with a client interface through a network in accordance with some embodiments.

FIG. 4 sets forth a flowchart illustrating the steps of generating a report including therapeutic recommendations for a subject based on mutations identified in the subject's nucleic acid sample, in accordance with some embodiments.

FIG. 5A illustrates a data visualization region including a plurality of panels, each panel populated with clinically relevant information pertaining to identified gene variants or interacting gene variants in accordance with some embodiments.

FIG. 5B illustrates a representation populated within clinically relevant information in accordance with some embodiments.

FIG. 5C illustrates a representation populated within clinically relevant information in accordance with some embodiments.

FIG. 5D illustrates a representation populated within clinically relevant information in accordance with some embodiments.

FIG. 5E illustrates a visualization of clinically relevant information pertaining to individual identified gene variants or interacting identified gene variants in accordance with some embodiments.

FIG. 5F illustrates representations providing more detailed clinically relevant information as compared with representation 510 of FIG. 5A in accordance with some embodiments.

FIG. 5G illustrates a representation populated within clinically relevant information in accordance with some embodiments.

FIG. 5H illustrates a representation populated within clinically relevant information in accordance with some embodiments.

FIG. 6A illustrates a method of remotely generating one or more visualizations or reports including clinically relevant information in accordance with some embodiments.

FIG. 6B illustrates a method of reviewing a draft report for approval in accordance with some embodiments.

FIG. 7 illustrates a method of retrieving and/or reviewing a finalize report in accordance with some embodiments.

FIG. 8A illustrates a representation populated within clinically relevant information including representative indicia in accordance with some embodiments.

FIG. 8B illustrates a representation populated within clinically relevant information including representative indicia in accordance with some embodiments.

FIG. 9A illustrates a representation populated within clinically relevant information, including therapeutic recommendations arranged within a column, in accordance with some embodiments.

FIG. 9B illustrates a representation populated within clinically relevant information, including therapeutic recommendations arranged within a column, in accordance with some embodiments.

FIG. 10 illustrates a data visualization region including a plurality of panels, each panel populated with clinically relevant information pertaining to identified genes and where interacting gene variants are identified (interaction summaries) before the individual panels in accordance with some embodiments.

FIG. 11 provides a flowchart which illustrates the steps of displaying one or more representations or visualizations of clinically relevant information in accordance with some embodiments.

FIG. 12 provides a flowchart which illustrates the steps of displaying one or more representations or visualizations of clinically relevant information in accordance with some embodiments.

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of” or “exactly one of.” “Consisting essentially of,” when used in the claims, shall have its ordinary meaning as used in the field of patent law.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “filtering” refers to altering one or more data sets. Filtering can mean keeping, adding, subtracting, or adding back data points (e.g. clinical information) from a data set. Filtering can mean masking one or more data points within the data set. Filtering can mean unmasking data points in a data set. In some embodiments filtering is an iterative process. In some embodiments filtering is performed with one or more filters. In some embodiments data points removed or masked by one filter are added back or unmasked by a second filter. In some embodiments filtering is performed on a list of variants. A filtered dataset can be smaller or larger than the original dataset. In some embodiments the filtered dataset comprises data points not removed from the original data set.

As used herein, the terms “read depth” or “sequencing depth” refer to the number of times a sequence has been sequenced (the depth of sequencing). As an example, read depth can be determined by aligning multiple sequencing run results and counting the start position of reads in non-overlapping windows of a certain size (for example, 100 bp). Copy number variation can be determined based on read depth using methods known in the art. For example, using a method described in Yoon et al., Genome Research 2009 September; 19(9): 1586-1592; Xie et al., BMC Bioinformatics 2009 Mar. 6; 10:80; or Medvedev et al., Nature Methods 2009 November; 6(11 Suppl): S13-20.

As used herein, the terms “sequence data” or “sequencing data” refer to any sequence information on nucleic acid molecules known to the skilled person. The sequence data can include information on DNA or RNA sequences, modified nucleic acids, single strand or duplex sequences, or alternatively amino acid sequences, which have to converted into nucleic acid sequences. The sequence data may additionally comprise information on the sequencing device, date of acquisition, read length, direction of sequencing, origin of the sequenced entity, neighboring sequences or reads, presence of repeats or any other suitable parameter known to the person skilled in the art. The sequence data may be presented in any suitable format, archive, coding or document known to the person skilled in the art.

As used herein, the term “user interface” refers to the interface that allows the user, for example end users such as geneticists, to input commands and data and receive results, such as a graphical user interface (GUI). The terms “user interface” and “graphical user interface” are used interchangeably herein.

As used herein, the terms “variant” or “genetic variant” refer to an alternative form of a gene, a genomic sequence, or portions thereof. A variant can also be referred to on a protein or RNA level, corresponding to the genomic change. In some embodiments, a variant causes changes of amino acids in a protein sequence but can also impact the function or activity of a protein or cell otherwise, such as in terms of RNA splicing, translation, or on other levels of transcription or translation regulation. Variant” can also refer to a polypeptide in which the sequence differs from the sequence most prevalent in a population at a position that does not change the amino acid sequence of the encoded polypeptide (i.e., a conserved change). Genetic variant polypeptides can be encoded by a risk haplotype, encoded by a protective haplotype, or can be encoded by a neutral haplotype. Genetic variant polypeptides can be associated with risk, associated with protection, or can be neutral. Non-limiting examples of genetic variants include frameshift, stop gained, start lost, splice acceptor, splice donor, stop lost, missense, splice region, synonymous and copy number variants. Non-limiting types of copy number variants include deletions and duplications.

As described in further detail herein, the present disclosure provides a system, method, and interface to facilitate the interpretation of one or more gene variants (e.g. variants in the same or different genes) identified in sequence data, such as sequence data obtained from a human patient's nucleic acid sample (e.g. a sample derived from a tumor). At times, looking to a single genomic variant may not be completely determinative, e.g. looking for a predictive therapeutic outcome based on the presence (or absence) or a single genomic variant may not be optimal in that it has a limited context. By considering other gene variants, such as those that would have a positive or negative contribution to the therapeutic outcome, improved guidance may be provided to the practicing clinician such that optimal treatment, in view of a plurality of gene variants, may be administered. In this way, the present disclosure provides a system and method for interpreting the clinical significance of how certain variants relate or interact with one another and visualizing or otherwise reporting the clinical significance of the interaction and/or a recommendation to a user or clinician based on the presence of the interacting genes. The systems and methods described herein are efficient, enabling rapid analysis and identification of gene variants from obtained sequence data, and the quick retrieval of relevant curated data associated with one or more variants (including interacting gene variants). Moreover, the systems and methods described herein enable the visualization and reporting of the gene variants (such as to a computer display or to a user operating a computing device), as well as information pertaining to interactions between gene variants, in an optimal and organized manner. In some embodiments, the systems and methods enable the rapid interpretation of visualizations including clinically relevant information by users (e.g. medical professionals, geneticists, doctors, etc.), whereby the rapid interpretation facilitates quick and accurate medical decisions which may lead to improvements in patient care. In some embodiments, the visualizations provide more information to a user in a simplified manner, allowing for a more rapid review of the clinically relevant information, and to facilitate efficient review of data pertaining to gene interactions.

In some embodiments, the present disclosure provides systems comprising an improved user interface which is adapted to enable the visualization of clinically relevant information pertaining to gene variants and interacting gene variants, including therapeutic recommendations. In some embodiments, the improved user interface facilitates the contemporaneous visualization of clinically relevant information pertaining to individual gene variants and the visualization of clinically relevant information pertaining to an interaction between gene variants, including therapeutic recommendations. It is believed that the visualization(s), through the improved user interface, facilitates the rapid interpretation of clinically relevant information by a medical professional such that decisions regarding patient care may be made accurately and efficiently. It is further believed that the systems and methods disclosed herein provide medical professionals with visualizations that enable quick, accurate, and efficient review and interpretation of clinically relevant information.

In some embodiments, the present disclosure provides a data visualization system which enables a user to review a data set of retrieved genomic variants and corresponding clinical information. In some embodiments, the data visualization system provides a graphical user interface to interactively review and interpret clinical data. The data visualization system also enables a user to appreciate the interrelations between multiple gene variants found in a subject's nucleic acid sample. The visualization of information, such as through organized panels (such as user interactive panels), facilitates the conveyance of relevant clinical information to allow a user to effectively interpret the presented clinical data and make any changes (e.g. annotations, reclassifications, etc.) necessary prior to reporting of the retrieved data. The data visualization system may also be utilized to generate a report summarizing the reviewed and interpreted clinical information.

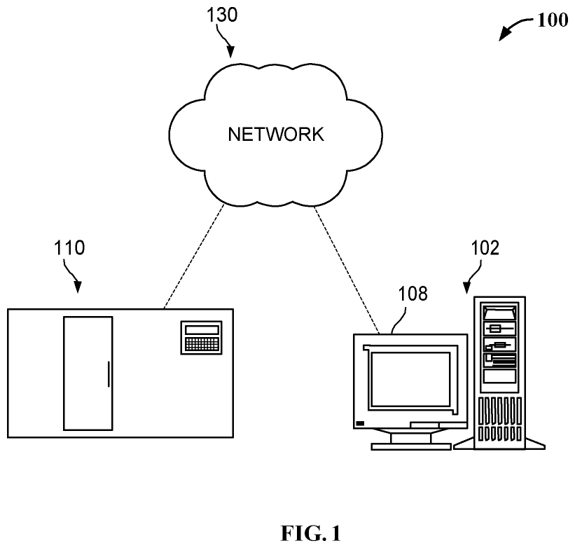

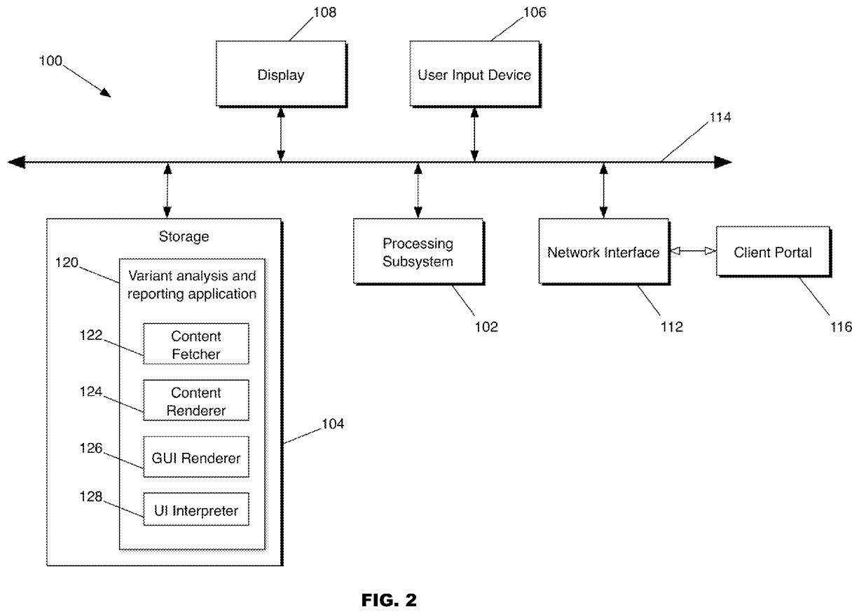

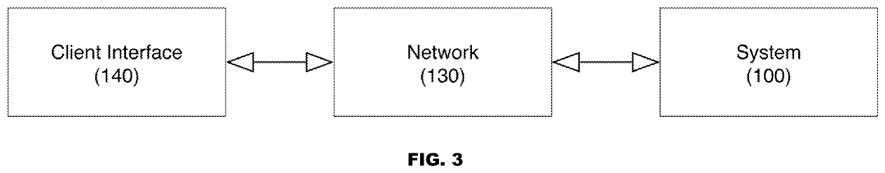

In some embodiments, systems of the present disclosure are adapted to facilitate the interpretation and reporting of sequence data obtained from a subject (e.g. a human patient in need of a therapeutic treatment). In some embodiments, sequence data is derived from a sequencing operation using a sequencing device and the sequence data is stored in a database, such as a networked database, for later interpretation and reporting. For example, sequence data may be derived using a sequencing device and stored in a file (e.g. in variant call format, BED format, or BEDPE format) located on a storage subsystem 104 or a networked server, whereby the file may be later retrieved for gene variant identification, retrieval of relevant clinical information pertaining to the identified gene variants (or interaction between multiple gene variants), visualization, and/or reporting (see FIG. 1). In some embodiments, software, such as a variant analysis and reporting application, is run directly on a system and the sequence data is retrieved from the networked server for interpretation and reporting (see FIG. 2). In other embodiments, software, such as a variant analysis and reporting application is run on a remote system and a client interface or client portal is used to access the system, and whereby sequence data may be uploaded to the remote system through the client interface for interpretation and reporting (see FIGS. 2 and 3).

The systems and methods provided herein can be applied to the interpretation and reporting of any variant in any gene of interest. Exemplary variants include single nucleotide polymorphisms, point mutations, insertions, deletions, and translocations. For example, the systems and methods may be utilized to interpret and report on variations detected within an EGFR gene or a BRCA1 gene, including multiple variations detected within any specific gene (e.g. EGFR p.L858R and EGFR p.T790M).

FIG. 1 sets forth a system 100 (a computer or computing device) including a sequencing device 110 communicatively coupled to a processing subsystem 102. The sequencing device 110 can be coupled to the processing subsystem 102 either directly (e.g., through one or more communication cables) or through one or more wired and/or wireless networks 130. In some embodiments, the processing subsystem 102 may be included in or integrated with the sequencing device 110. In some embodiments, the system 100 may include software to command the sequencing device 110 to perform certain operations using certain user configurable parameters, and to send resulting sequencing data acquired to the processing subsystem 102 or a storage subsystem (e.g. a local storage subsystem or a networked storage device). In some embodiments, either the processing subsystem 102 or the sequencing device 110 may be coupled to a network 130. In some embodiments, a storage device is coupled to the network 130 for storage or retrieval of sequence data, patient information, and/or other tissue data. The processing subsystem 102 may include a display 108 and one or more input devices (not illustrated) for receiving commands from a user or operator (e.g. a technician or a geneticist). In some embodiments, a user interface is rendered by processing subsystem 102 and is provided on display 108 to (i) facilitate the analysis, interpretation, and/or reporting of sequencing data and/or patient data; (ii) to retrieve data from a sequencing device; or (iii) to retrieve sequencing data, patient information, or other clinical information from a database, such as one available through a network. In some embodiments, the network 130 enables access to a processing subsystem 102 and/or a sequencing device 110 remotely, such as through a client interface or client portal (not illustrated). In this way, a remote user may access the processing subsystem 102 such that variant analysis and reporting software may be run remotely on the processing subsystem 102. In some embodiments, the client interface or client portal may also enable the retrieval of stored reports after analysis and interpretation of sequence data. In some embodiments, the client interface or client portal may facilitate the transfer of sequence data to the processing subsystem 102 or any attached local or networked storage device communicatively coupled therewith. Methods of sequencing a sample are described in PCT Publication Nos. WO/2017/123316 and WO/2017/181134, the disclosures of which are hereby incorporated by reference herein in their entireties.

FIG. 2 is a block diagram of a system 100 according to an embodiment of the present disclosure. System 100 can be implemented using any type of user-operable computing device, including desktop computers, laptop computers, tablet computers, handheld devices (e.g., smart phones, media players), and so on. System 100 can include a number of interconnected components such as processing subsystem 102, storage subsystem 104, user input device 106, display 108, and network interface 112 communicating via bus 114, as discussed in more detail below. In some embodiments, the system 100 depicted in FIG. 2 may be accessed remotely, e.g. one or more remote users may access system 100, such as over a network, such that sequence data stored within storage subsystem 104 or sequence data remotely uploaded to the storage subsystem 104 may be interpreted, visualized, and/or reported.

Processing subsystem 102 can include a single processor, which can have one or more cores, or multiple processors, each having one or more cores. In some embodiments, processing subsystem 102 can include one or more general-purpose processors (e.g., CPUs), special-purpose processors such as graphics processors (GPUs), digital signal processors, or any combination of these and other types of processors. In some embodiments, some or all processors in processing subsystem can be implemented using customized circuits, such as application specific integrated circuits (ASICs) or field programmable gate arrays (FPGAs). In some embodiments, such integrated circuits execute instructions that are stored on the circuit itself. In other embodiments, processing subsystem 102 can retrieve and execute instructions stored in storage subsystem 104, and the instructions may be executed by processing subsystem 102 regardless of whether a user accesses the system locally or remotely, such as through client portal 116. By way of example, processing subsystem 102 can execute instructions to receive and process variant data (e.g. in variant call format) stored within a local or networked storage system and display identified gene variants from the input variant data together with retrieved clinical summaries and/or therapeutic recommendations relevant to the identified gene variants.

Storage subsystem 104 can include various memory units such as a system memory, a read-only memory (ROM), and a permanent storage device. A ROM can store static data and instructions that are needed by processing subsystem 102 and other modules of system 100. The permanent storage device can be a read-and-write memory device. This permanent storage device can be a non-volatile memory unit that stores instructions and data even when system 100 is powered down. In some embodiments, a mass-storage device (such as a magnetic or optical disk or flash memory) can be used as a permanent storage device. Other embodiments can use a removable storage device (e.g., a flash drive) as a permanent storage device. The system memory can be a read-and-write memory device or a volatile read-and-write memory, such as dynamic random access memory. The system memory can store some or all of the instructions and data that the processor needs at runtime.

Storage subsystem 104 can include any combination of non-transitory computer readable storage media including semiconductor memory chips of various types (DRAM, SRAM, SDRAM, flash memory, programmable read-only memory) and so on. Magnetic and/or optical disks can also be used. In some embodiments, storage subsystem 104 can include removable storage media that can be readable and/or writeable; examples of such media include compact disc (CD), read-only digital versatile disc (e.g., DVD-ROM, dual-layer DVD-ROM), read-only and recordable Blu-ray® disks, ultra-density optical disks, flash memory cards (e.g., SD cards, mini-SD cards, micro-SD cards, etc.), and so on. In some embodiments, data and other information (e.g. sequencing data, patient information, clinical data, tissue data, curated data, etc.) can be stored in one or more remote locations, e.g., cloud storage, and synchronized with other the components of system 100. The storage subsystem 104 may include a database of curated information. When the terms “memory” or “a memory” are used herein, they may refer to one or more memories, such as a plurality of memories.

In some embodiments, storage subsystem 104 can store one or more software programs to be executed by processing subsystem 102, such as a variant analysis and reporting application 120. “Software” refers generally to sequences of instructions that, when executed by processing subsystem 102, cause system 100 to perform various operations, thus defining one or more specific machine implementations that execute and perform the operations of the software programs. Thus, “software” can also include firmware or embedded applications or any other type of instructions readable and executable by processing subsystem 102. Software can be implemented as a single program or a collection of separate programs or program modules that interact as desired. In some embodiments, programs and/or data can be stored in non-volatile storage and copied in whole or in part to volatile working memory during program execution. From storage subsystem 104, processing subsystem 102 can retrieve program instructions to execute and data to process in order to execute various operations including operations described below. An example of software includes a variant analysis and reporting application 120. The software may be run locally on system 100 but accessed and/or controlled remotely, such as through a client portal 116. For example, an instance of the software may be run locally on system 100 but a remote operator may access the software by means of a network connected client portal 116 such that files (e.g. those that include variant data, such as in variant call format, BED format, or BEDPE format) may be uploaded to the storage subsystem 104 and whereby the remote user may control the instance of the software such that the interpretation, visualization, and/or reporting of a subject's variant data may be performed.

A user interface can be provided to a display device 108, and/or and one or more other user output devices (not shown). The user interface may include, for example, menu bars, a metadata pane, toolbars, drop-down menus, and visual representations of clinically relevant data. Input devices 106 can include any device via which a user can provide signals to system 100; system 100 can interpret the signals as indicative of particular user requests or information. In some embodiments, the representations provided by the user interface facilitate interaction with a user (either remote or local). For example, the user may select certain user configurable settings or parameters, such that retrieved data sets may be altered in accordance with the selection of the user configurable settings or parameters. In various embodiments, input devices 106 can include any or all of a keyboard touch pad, touch screen (e.g., a touch-sensitive overlay on a display surface of display 108), mouse or other pointing device, scroll wheel, click wheel, dial, button, switch, keypad, microphone, and so on.

Display 108 can display images (e.g. representations or visualizations including a one or more of a plurality of gene variants and the interactions between any number of the plurality of gene variants) generated by system 100 and can include various image generation technologies, e.g., a cathode ray tube (CRT), liquid crystal display (LCD), light-emitting diode (LED) including organic light-emitting diodes (OLED), projection system, or the like, together with supporting electronics (e.g., digital-to-analog or analog-to-digital converters, signal processors, or the like). Some embodiments can include a device such as a touchscreen that function as both input and output device. In some embodiments, other user output devices can be provided in addition to or instead of display 108.

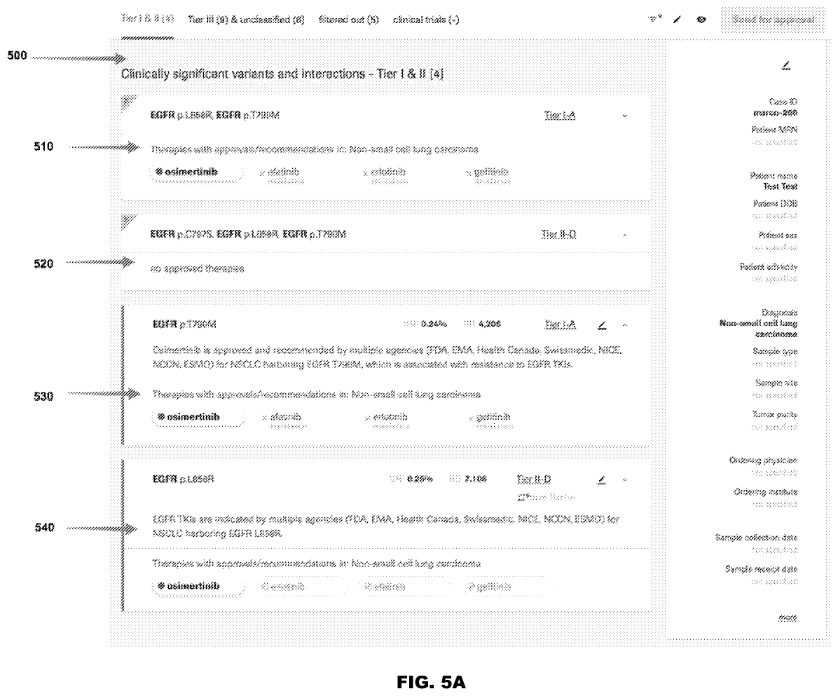

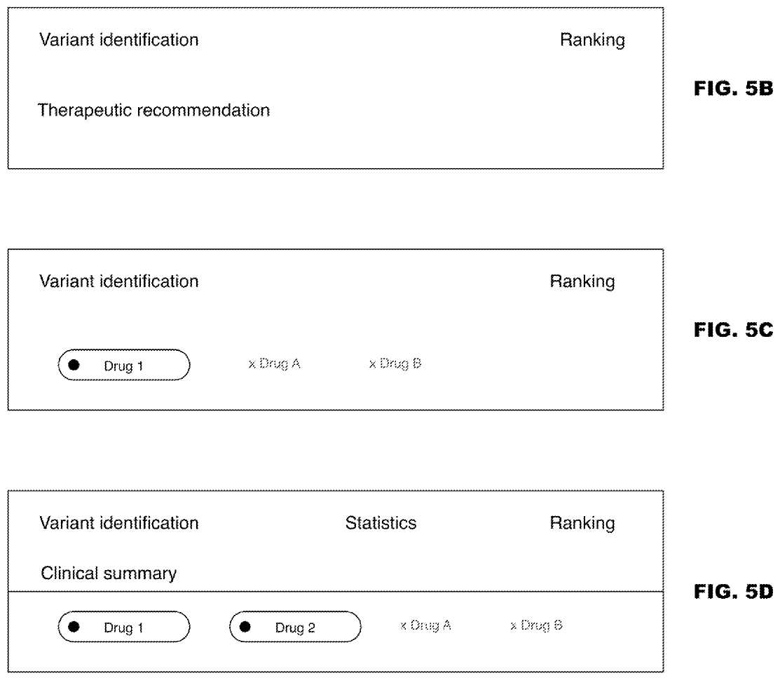

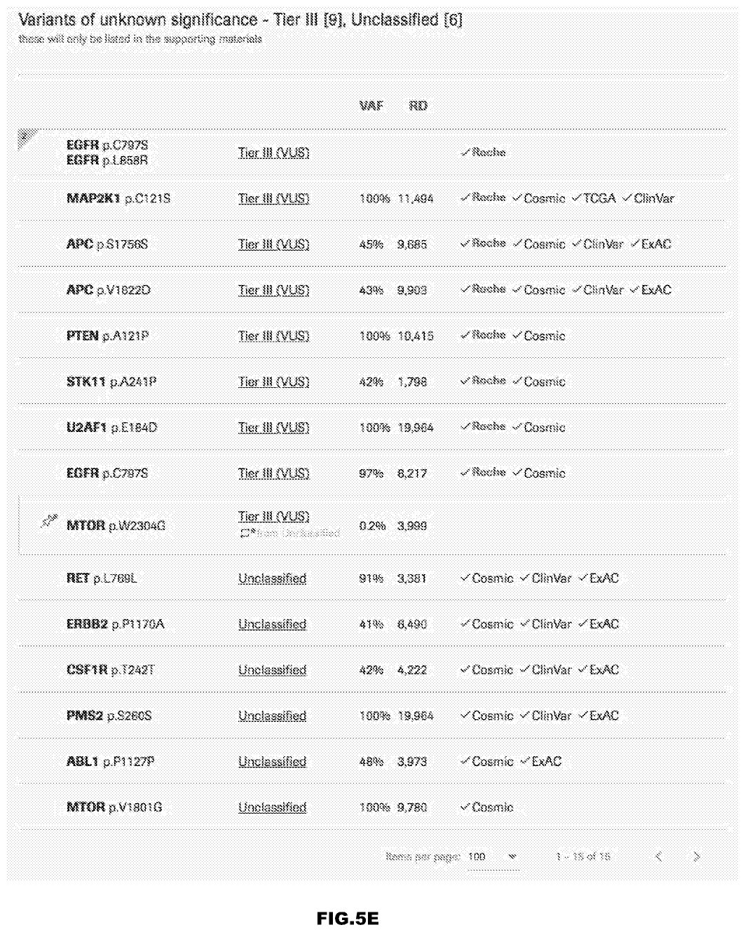

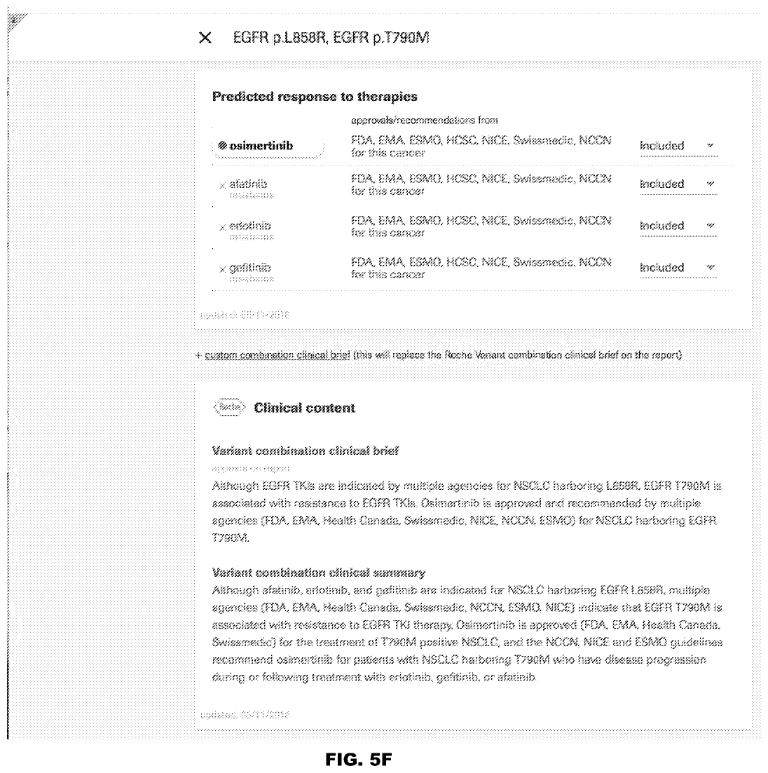

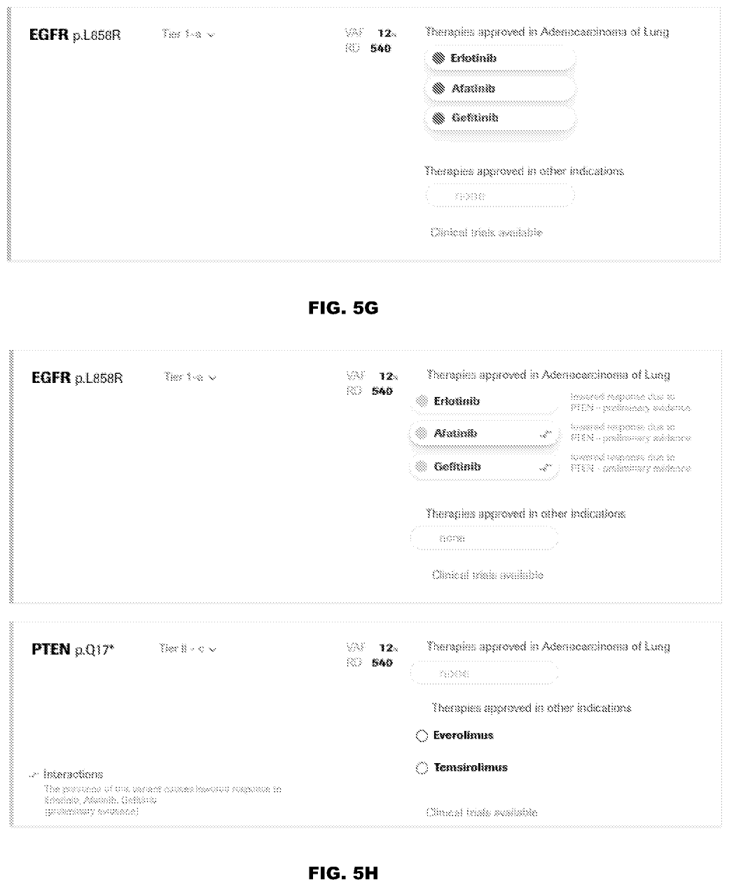

In some embodiments, the user interface can provide a graphical user interface, in which visible image elements in certain areas of display 108 are defined as active elements, interactive elements, or control elements that the user selects using user input devices 106. For example, the user can manipulate a user input device 106 to position an on-screen cursor or pointer over the control element, then “click” a button to indicate the selection (the selection sending signals to perform a designated action or routine). For example, the user can manipulate the user input device 106 to select an icon within the user interface which would effectuate the initiation of an operation, e.g. initiate a filtering operation, initiate the generation of a report, initiate the annotation of one or more displayed visualizations or representations. By way of another example, the user may click on a representation (e.g. a panel) within a rendered data visualization region (see 500 of FIG. 5A) such that a new window or new user interface screen is presented which provides further details as to clinically relevant information relating to one or more identified gene variants or the interaction between at least two gene variants (see FIG. 5F).

In some embodiments, the user can manipulate the user input device 106 so as to interact with a series of user configurable options such as icons, buttons, context menus, dropdown menus, toggle switches, etc. so as to define a set of user configurable parameters. Selection of the user configurable parameters could be used to, for example, alter datasets. For example, and as disclosed further herein, the user may select certain user configurable filter parameters, such as parameters displayed within a filter configuration submenu or pop-up window within the user interface, whereby signals generated by the selection may cause certain variant data (such as a particular patient's genomic data embedded within a variant call format data file) to be included or excluded from a report based on cutoff values selected for certain user selectable fields/criteria. By way of another example, the user input device 106 may be utilized such that boxes may be checked in a filter configuration panel to enable or disable searching within specific databases or curated collections. By way of yet another example, values (e.g. percentage values) may be provided by an operator through input device 106 and filtering may be initiated based on the user configurable values entered.

Alternatively, the user can touch the control element (e.g., with a finger or stylus) on a touchscreen device. In some embodiments, the user can speak one or more words associated with the control element (the word can be, e.g., a label on the element or a function associated with the element). In some embodiments, user gestures on a touch-sensitive device can be recognized and interpreted as input commands; these gestures can be, but need not be, associated with any particular area on display 108. Other user interfaces can also be implemented.

Network interface 112 may provide data communication capability for system 100. In some embodiments, network interface 112 can include radio frequency (RF) transceiver components for accessing wireless voice and/or data networks (e.g., using cellular telephone technology, advanced data network technology such as 3G, 4G or EDGE, 5G, WiFi (IEEE 802.11 family standards), or other mobile communication technologies, or any combination thereof, GPS receiver components, and/or other components. In some embodiments, network interface 112 can provide wired network connectivity (e.g., Ethernet) in addition to or instead of a wireless interface. Network interface 112 can be implemented using a combination of hardware (e.g., antennas, modulators/demodulators, encoders/decoders, and other analog and/or digital signal processing circuits) and software components. Network interface 112 may facilitate remote access to system 100, such as through a client portal 116 (for example, a remote user may access system 100 through a remote computer and the remote computer interacts with system 100 through the network interface 112). In some embodiments, the client portal 116 is a stand-alone application that is run by a remote user on a remote computer or other computing device. In other embodiments, the client portal 116 is a web-browser running on the remote computer or other computing device which accesses system 100 through a network.

Bus 114 can include various system, peripheral, and chipset buses that communicatively connect the numerous components of system 100. For example, bus 114 can communicatively couple processing subsystem 102 with storage subsystem 104. Bus 114 can also connect to input devices 106 and display 108. Bus 114 can also couple processing subsystem 102 to a network through network interface 112. In this manner, system 100 can be connected to a network of multiple computer systems (e.g., a local area network (LAN), a wide area network (WAN), an Intranet, or a network of networks, such as the Internet. The skilled artisan will appreciate that additional components may be connected to bus 114, such as a sequencing device, a scanning device, a tissue processing system, etc.

Some embodiments include electronic components, such as microprocessors, storage and memory that store computer program instructions in a computer readable storage medium. Many of the features described herein may be implemented as processes that are specified as a set of program instructions encoded on a computer readable storage medium. When these program instructions are executed by one or more processing units, they cause the processing unit(s) to perform various operation indicated in the program instructions. Examples of program instructions or computer code include machine code, such as is produced by a compiler, and files including higher-level code that are executed by a computer, an electronic component, or a microprocessor using an interpreter.

Through suitable programming, processing subsystem 102 can provide various functionalities for system 100. For example, processing subsystem 102 can execute a variant analysis and reporting application 120 having a user interface; or can execute a viewer application (e.g. a web browser) or other viewer window enabling review and/or analysis of clinical reports, including publicly available clinical reports (e.g. observations of correlations between therapeutic efficacy and a particular variant). The variant analysis and reporting application 120 can provide various functionality such as the ability to retrieve and display content from a database, such as a local database or a networked database (e.g. databases include those having curated information; summaries of curated information; reference, citations, or links to other databases or sources for additional clinical information; etc.).

In some embodiments, the variant analysis and reporting application 120 incorporates various interoperating modules (e.g., blocks of code) that, when executed by one or more processors within the processing subsystem 102, implement aspects of the interface operation. For example, variant analysis and reporting application 120 can include a content fetcher 122, a content renderer 124, a GUI renderer 126, and a UI interpreter 128.

In some embodiments, content fetcher 122 can include instructions for interacting with (e.g. accessing) a local database (e.g. storage subsystem 104) or with network interface 112 to fetch or otherwise retrieve content items, such as sequencing data, gene variant data, patient data, predicted therapeutic response data, clinical content (e.g. clinical briefs, clinical summaries, etc.), and biological content (e.g. gene biological summaries, variant functional summaries, etc.). In some embodiments, the content fetcher 122 is configured to obtain a plurality of gene variants from one or more memories communicatively coupled to the system (e.g. a computer device). In some embodiments, the plurality of gene variants are derived from sequence data derived from a patient sample. In some embodiments, data may also be retrieved from public databases. In some embodiments, the content fetcher 122 may retrieve information from one or more curated databases.

In some embodiments, content renderer 124 can include instructions for interpreting fetched content items from one or more sources and then populating or delivering the rendered content to representations generated by the GUI rendered 126. For example, content renderer 124 may populate one or more rendered panels (described herein) with identified gene variants (or interacting gene variants) and corresponding content retrieved by content fetcher 122. In some embodiments, content renderer 124 provides a quantity of information based on the type visualization being rendered by the GUI rendered 126, and the information may include summaries or abstracts of retrieved content and/or brief summaries of therapeutic interventions. For example, if GUI renderer 126 provides a series of panels, the content fetcher 124 pay populate the series of panels with summary information or “high level” information, such as according to user preferences. However, if the GUI renderer 126 renders windows for the conveyance of more granular information, the content renderer 124 will populate those windows with a comparatively greater level of information as compared with the information populated into the panels. In some embodiments, the content renderer 124 is configured to display on a display screen a plurality of representations or visualizations comprising data pertaining to one or more gene variants and/or an identified clinically relevant interaction between at least two gene variants (e.g. first, second, third, or nth representations or visualizations).

In some embodiments, the content renderer 124 is configured to display on a display screen a first representation comprising data pertaining to an identified clinically relevant interaction between at least two gene variants of a plurality of gene variants. In some embodiments, the content renderer 124 is configured to display on a display screen a second representation, along with the aforementioned first representation, comprising clinically relevant information pertaining to a first of the at least two gene variants for which an interaction was identified. In some embodiments, the content renderer 124 is configured to display on a display screen a third representation, along with the aforementioned first and second representations, comprising clinically relevant information pertaining to a second of the at least two gene variants for which an interaction was identified. In some embodiments, the first, second, and third representations are displayed contemporaneously.

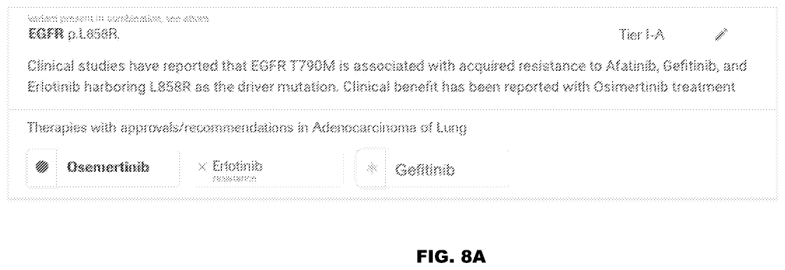

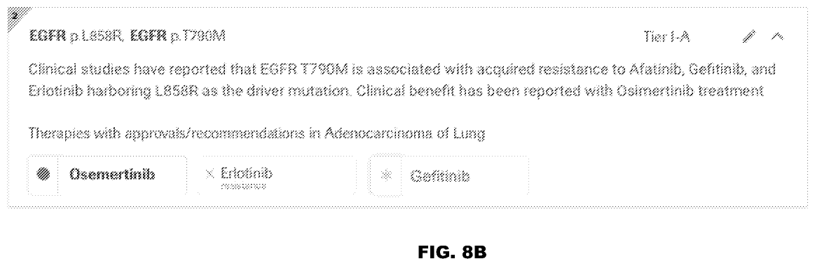

In some embodiments, GUI renderer 126 creates graphical user interface (GUI) elements to be presented to the user along with the content items rendered by content renderer 124 or other system modules. GUI renderer 126 can include code defining the location and appearance of GUI elements, such as a menu bar, window overlays, configuration panels, pop-up menus, annotation entry windows, panels, etc., each of which may be interactive elements. In some embodiments, GUI renderer 126 can generate panels having various sizes to accommodate the presentation of clinically relevant information to an operator, whereby the panels may include certain indicia to expedite review by the operator (see, e.g. FIGS. 8A and 8B). The GUI renderer 126 may also provide a workspace such that a user or operator may interact with the system 100 and/or control certain aspects of the data analysis and the reporting of retrieved clinical, biological, and/or therapeutic information.

UI interpreter 128 can receive user input, e.g., via a user input device 106, and can interpret the input to determine actions to be performed by sequencing data analysis and reporting application 120. For example, UI interpreter 28 can determine which GUI element (e.g. an icon, or a selectable item in menu, context menu, dropdown list, buttons, a panel, etc.) the user selected and initiate the corresponding action (e.g., add an annotation, display additional content information, generate a final report for exporting).

By way of example, the GUI renderer 126 may generate a series of panels which may be populated (e.g. with content renderer 124) with information retrieved by the content fetcher 122. Examples of panels are illustrated in FIG. 5A. These panels may be interactive panels. If a user clicks on any particular panel (e.g. panel 510 of FIG. 5A) (such as interpreted by UI interpreter 128), the GUI renderer 126 may render a new screen including a plurality of windows or representations (see FIG. 5F). Subsequently, the plurality of rendered windows or representations are then populated with information by content renderer 124 (such as with information retrieved by content fetcher 122). Content that may be retrieved (e.g. from one or more databases, including curated databases and/or public databases) and populated may include therapeutic options, clinical briefs and summaries, biological and functional summaries, classification information, statistics, and/or graphical variant alignment representations. In this way, the panels, such as those depicted in FIG. 5A, may provide a high level of information (e.g. summary information or recommendations) to the user based on the presence or absence of certain gene variants or combinations of gene variants. Comparatively, the windows and representations, such as those depicted in FIG. 5F, provide a greater level of detail to the user as compared with the information provided in the panels of FIG. 5A, and this greater level of detail may be specifically requested by the user as needed, such as to better appreciate and/or interpret the correlation between one or more identified gene variants, retrieved clinically relevant information, and provided therapeutic recommendations/information.

It will be appreciated that system 100 is illustrative and that variations and modifications are possible. Further, while system 100 is described with reference to particular blocks, it is to be understood that these blocks are defined for convenience of description and are not intended to imply a particular physical arrangement of component parts. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software. Variant analysis and reporting application 120 is also illustrative, and specific implementations may include more or fewer modules than described herein. Moreover, while a particular module may be described as performing a particular function, such descriptions are not intended to imply a particular function performed by the module or a particular set of instructions included within such module.

FIG. 3 depicts a client interface 140 in communication with a network 130 and a system 100 (such as the systems depicted in FIGS. 1 and 2). The client interface 140 may be a stand-alone application (e.g. stand-alone variant analysis and reporting software) or a web-browser or other interface software which allows remote access to software 120. For example, client interface 140 allows a remote operator to log-into system 100 (such as the system depicted in FIGS. 1 and 2) and access stored sequence data (such as data stored in storage subsystem 104 or other network attached storage device) or sequence data uploaded to system 100 for processing. Of course, the client interface 140 may include any of the software modules described herein. In this way, a remote user may remotely interact with elements of the system (e.g. configurable elements) such that sequence data may be interpreted and reported (e.g. a geneticist may select user configurable parameters, such as filtering parameters, review curated information retrieved by the system in response to a filtering operation, annotate information, selectively add or remove information from select databases, and/or generate a report for review). In some embodiments, system 100 may receive a request from a remote user through the client interface 140, where the request may include a request for a report to be generated based on uploaded sequence data (e.g. a remote user may upload sequence data and a different operator may perform the steps necessary to interpret the uploaded sequence data and generate an appropriate report). Alternatively, a remote operator may use client interface 140 to review generated reports (e.g. a clinician may use the client interface 140 to review generated reports for a patient and prescribe or recommend therapy based upon recommendations provided within the report). In yet other embodiments, the client interface 140 may serve as a patient portal enabling a patient to view a report based on a previously collected and sequenced sample.

The present disclosure also provides methods of identifying gene variants (or interacting gene variants) and matching those identified gene variants to clinically relevant information. The matching of the identified gene variants with the clinically relevant information may be visualized, such to aid in the interpretation of information for inclusion within a report. In some embodiments, a report may be generated including curated clinical information relevant to one or more identified gene variants (or interacting gene variants), and the report may be generated based on the visualized information which is reviewed and/or interpreted by a user of system 100.

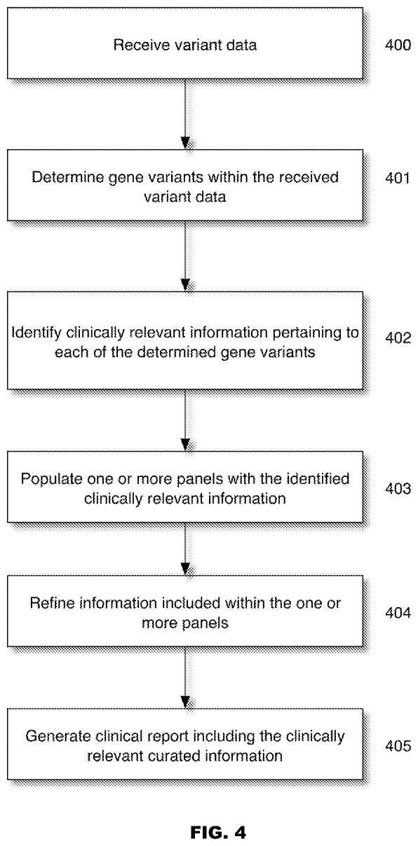

With reference to FIG. 4, variant data is first received by the system 100 (step 400). The variant data may be derived from a nucleic sample obtained from a subject, such as a human patient diagnosed with a particular disease or condition. In some embodiments, the variant data includes variants, or mutations, that are found in the nucleic acid sequence information that was obtained from a patient (i.e. from a patient's nucleic acid sample that has been sequenced with sequencing device 110). A mutation refers to a change in genetic information. As is known in the art, mutations include substitutions, insertions or deletions (INDELs), translocations, inversions, chromosomal abnormalities, and others.

In some embodiments, the variant data is provided in variant call format, which may be uploaded to or retrieved by system 100. In some embodiments, the data contained in the variant call format file represents the In some embodiments, the variant call format file includes, for example, eight columns, corresponding to data columns representing the chromosome (CHROM), a 1-based position of the start of the variant (POS), unique identifiers of the variant (ID), the reference allele (REF), a comma separated list of alternate non-reference alleles (ALT), a phred-scaled quality score (QUAL), site filtering information (FILTER) and a semicolon separated list of additional, user extensible annotation (INFO).

After the system 100 parses the variant data provided, variants in genes are determined (step 401). Clinically relevant information is then retrieved pertaining to each of the determined gene variants for an identified disease or condition (step 402). In some embodiments, the clinically relevant information is retrieved from a curated database of medical literature. In some embodiments, the curated database represents a distillation of the medical literature available (e.g. publicly available in peer-reviewed journal articles) and where the curated database is maintained, i.e. kept up-to-date as new literature becomes available.

In some embodiments, the curated database includes known gene variants and information pertaining those known gene variants. In some embodiments, the curated database correlates a particular gene variant with an approved or recommend therapy, combination therapy, or treatment protocol. In other embodiments, the curated database also includes summaries of biological and functional information pertaining to the gene variant. In other embodiments, the curated database further includes cross references or hyperlinks to source material, e.g. articles, data, or other information collected by the National Center for Biotechnology Information (NCBI). In some embodiments, the curated database further includes information pertaining to the gene variant location, i.e. chromosome location, position, and nucleotide change.

In some embodiments, the clinically relevant information for each gene variant (or group of gene variants) is retrieved by matching certain metadata fields within the curated database with the determined gene variants. In some embodiments, the “p. notation” or “c. notation” is used to map the determined gene variants to those within the database. In some embodiments, different matching may be performed to a geographic location or region (e.g. a treatment protocol or drug may not be approved in certain regions or countries). Clinically relevant information may then be identified based on the matching of database metadata with the patient's determined gene variants and diagnosed disease/condition, such as by using content fetcher 122.

For example, a patient's determined gene variants and the patient's diagnosed disease/condition may each be compared to metadata fields (e.g. variant metadata, biomarker metadata, disease type metadata, etc.) in a curated database. By way of a further example, assume that a patient has been diagnosed with non-small cell lung cancer. Further assume that the patient carries the mutation EGFR p.L858R. The patient's diagnosed disease (non-small cell lung cancer) and the determined mutation (EGFR p.L858R) are then compared to metadata fields in the curated database. A match is returned for an entry showing the EGFR p.L858R as it relates to non-small cell lung cancer. The skilled artisan will appreciate that the EGFR p.L858R mutation may be common for other diagnosed conditions, but a match will only be returned if both the determined gene variant and the diagnosed disease/condition are matched.

The skilled artisan will further appreciate that matches may also be made on more than one gene variant. For example, a database entry describing the interaction of the EGFR p.L858R and EGFR p.T790M mutations for patients diagnosed with non-small cell lung cancer will only be matched with a patient entry including the non-small cell lung cancer diagnosis entry and the entry of both the EGFR p.L858R and EGFR p.T790M mutations.

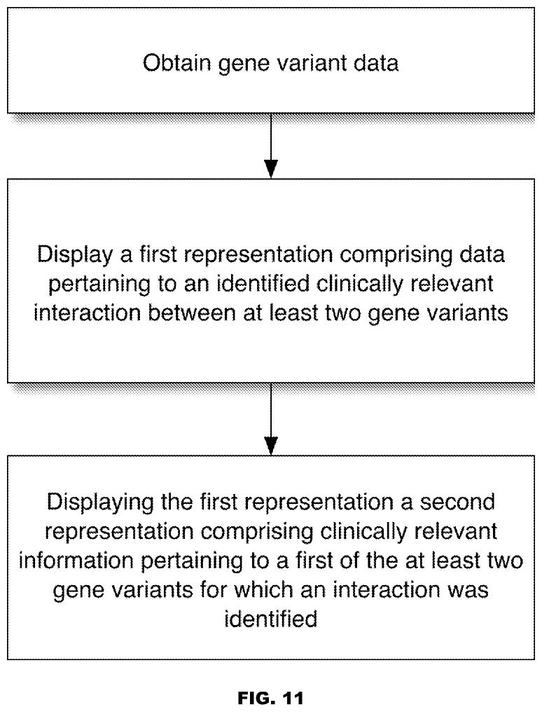

The clinically relevant information retrieved may then be visualized, such as through a user interface on display 108 (see FIGS. 11 and/or 12). In some embodiments, the visualizations enable the rapid and efficient review of clinically relevant information so as to enable quick, accurate, and informed decision making and improved patient care. In some embodiments, the graphical user interface includes a data visualization region (see 500 in FIG. 5A). In some embodiments, the GUI renderer 126 generates one or more representations within the data visualization region 500, which may each be individually populated with at least some of the retrieved information (e.g. a summary of the identified clinically relevant information pertaining to the patient's one or more determined gene variants). In some embodiments, the representations include graphical and/or textual information which the user may interpret, analyze, and/or interact with, e.g. the user may review the information within the representations and make changes to the information and/or add annotations to the information, such as described herein.

In other embodiments, the GUI renderer 126 generates one or more panels and then each of the one or more generated panels each may be individually populated with at least some of the clinically relevant information retrieved from the curated database (step 403) (see also FIG. 5A). In some embodiments, each of the panels are equally sized in one dimension and dynamically sizeable in another dimension, such as according to the amount of clinically relevant content retrieved and populated therein. In some embodiments, the panel may include a summary of gene variants identified and clinically approved or recommended therapies for treating a particular disease or condition in patients having the identified one or more gene variants (see, e.g. 510, 520, 530, or 540 of FIG. 5A). In some embodiments, the populated panel may include a summary of a published journal article or other source material from which the clinical relevant information was curated from (e.g. a one or two sentence summary). In some embodiments, the summary may be revealed or hidden depending on user preferences.

In some embodiments, the panels are interacting gene panels, such as 510 and 520 of FIG. 5A. In these examples, the interacting genes are identified, along with therapeutic recommendations. In some embodiments, the therapeutic recommendation is not to administer a particular drug or treatment protocol. In some embodiments, the therapeutic recommendation is a therapy sensitive for a disease of interest. Clinical summary information, while available for display within panels 510 and 520, is hidden based on user preferences (see, for comparison, FIG. 8B which shows a short summary of relevant clinical information).

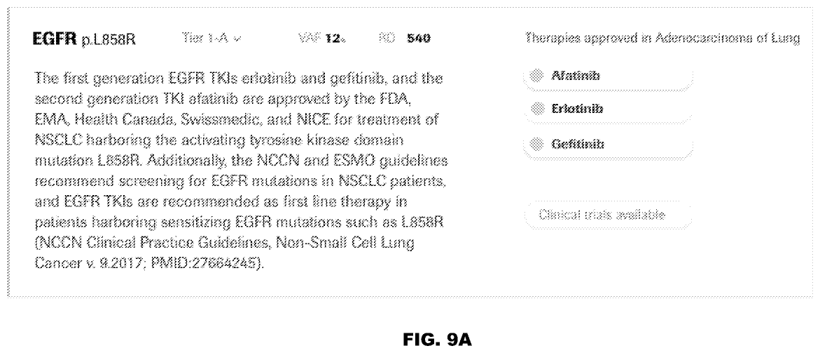

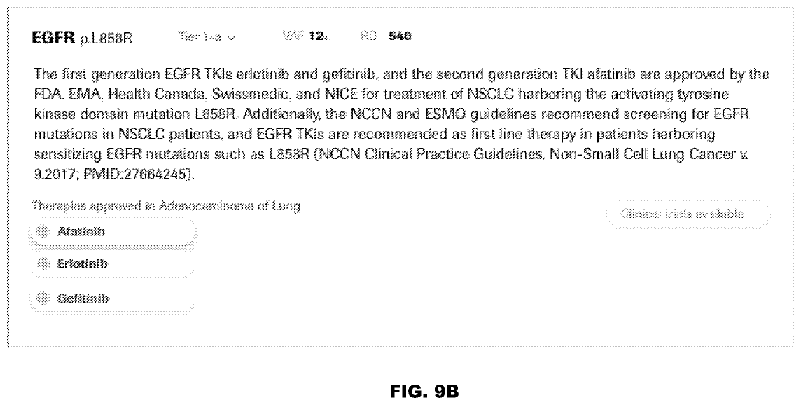

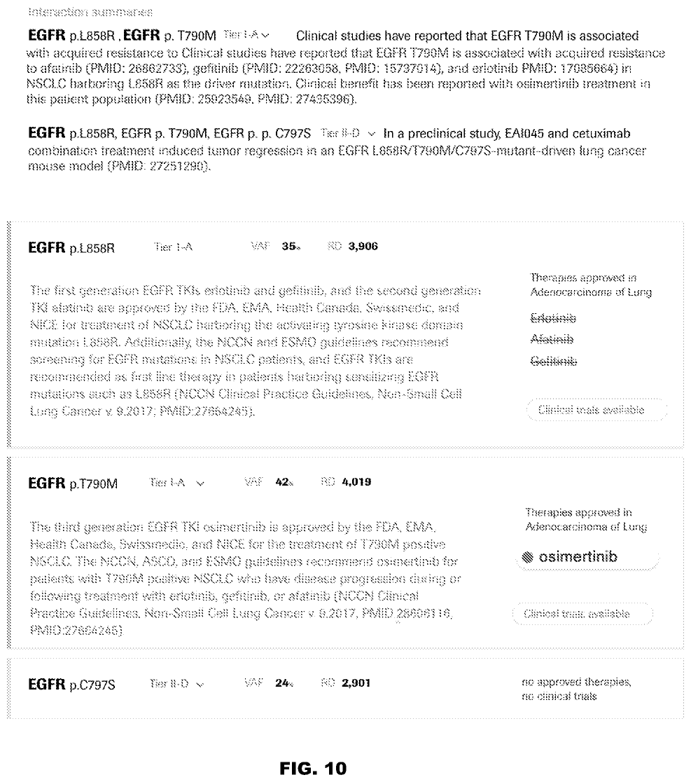

In other embodiments, the panels are individual gene panels, such as 530 and 540 of FIG. 5A. In these examples, the individual genes are identified, along with therapeutic recommendations, and a short clinical summary. The short clinical summary may be hidden depending on user preferences. In these examples, statistical information is also presented in the individual gene panels. In some embodiments, a classification rating is displayed in both the interacting gene panels and the individual gene panels. In some embodiments, a recommended therapeutic approach is presented within a stand-out box, such that the recommendation is more prominently displayed than those therapeutics which are not suggested for use. In some embodiments, the therapeutic recommendations appear in a second portion of the panel as in FIG. 5A. In some embodiments, the therapeutic recommendations are in-line with each other, i.e. arranged in a single row (see FIG. 5A). In other embodiments, the therapeutic recommendations are aligned in one or more columns (see FIGS. 9A and 9B). In some embodiments, interacting gene variants are not shown within panels, but rather set forth above respective individual gene panels, such as illustrated in FIG. 10.

In some embodiments, at least two representations are generated. In some embodiments, the at least two representations generated (i.e. first and second representations) are displayed within a single panel. In some embodiments, the single panel is an individual gene panel representation, as described herein. In some embodiments, a second representation of the at least two representations is visualized in a first portion of the individual gene panel representation and wherein a first representation of the at least two representations is visualized in a second portion of the individual gene panel representation.

In other embodiments, the at least two representations are displayed within separate panels. In some embodiments, a first representation of the at least two representations is displayed within an interacting gene panel (as described herein), and wherein a second representation of the at least two representations is displayed within an individual gene panel. In some embodiments, the individual gene panel further comprises the first representation and identification of the first of the at least two gene variants.

In some embodiments, the user may interact with the populated panels such that information may be modified, or annotations may be made. In some embodiments, the user may select a particular panel using user interface 106 and a new window, overlay, or screen may be prepared by the GUI renderer 126 and populated with retrieved information (see FIG. 5F). In some embodiments, the new window, overlay, or screen includes additional clinical information and/or citations to source material that was not included within the one or more generated panels (compare panel 510 of FIG. 5A with FIG. 5F). In some embodiments, the new window, overlay, or screen includes a plurality of non-overlapping boxes which may be individually populated with different information including therapeutic options, clinical briefs and summaries, biological and functional summaries, classification information, and/or statistics. In some embodiments, the user may interact with the boxes populated with the retrieved information such that the content may be altered by selecting certain user configurable parameters, or such annotations may be added.

In some embodiments, one or more panels are populated with clinically relevant information pertaining to the presence of multiple gene variants and/or their interaction with themselves or other gene variants (see, e.g. 501 of FIG. 5A). In some embodiments, the multiple genomic variants are known (e.g. from published literature sources or information in a curated database) to confer additional sensitivity or resistance to phenotypic effects of a particular genomic variant. In some embodiments, the curated database can include briefs or entire references that describe the relationships between genetic variations that demonstrate the additional sensitivity or resistance to phenotypic effects and the one or more panels may be populated with the information from the curated database that matches those multiple gene variants that interact with one another.

The skilled artisan will appreciate that the presence of multiple variants may have an effect on a clinical approach or clinical outcome. In some embodiments, the co-occurrence of one genetic variant with one or more other genetic variants may have a clinically significant effect thereby resulting in an alternation of a recommended or approved course of treatment. For example, a particular treatment option may be approved and/or indicated when a patient has a certain first gene variant. However, that same treatment option may not be an efficacious option in a patient having that first gene variant and also having a second gene variant, where the second gene variant reduces the clinical significance of having the first gene variant (i.e. there is a negative implication of having both the first and second gene variants on one or more treatment protocols, as compared with having only the first gene variant). By way of another example, and assuming that a single treatment option is approved and/or indicated when a patient has a first gene variant, the presence of a second gene variant may expand the treatment options, i.e. providing additional treatment options other than the single approved and/or indicated treatment option. In this second example, the presence of the second gene variant causes a positive effect. As such, any time the presence of a particular gene variant has a negative or positive interaction with another gene variant, a clinical approach, clinical treatment protocol, or clinical outcome may be modified based on the presence (or absence) of the interaction between the two or more gene variants.

By way of another example, a patient with non-small cell lung carcinoma may test positive for the EGFR p.L858R variant, which is typically associated with sensitivity to a plurality of targeted therapies. However, the assessment may be modified by the presence one or more other variants (or lack of variants) in EGFR or, for that matter, in other genes that are known to or believed to modify patient response to one of the plurality of targeted therapies (again, according to published literature or other curated sources). According to this specific example, the assessment of which targeted therapy should be utilized is modified by the positive presence of EGFR p.T790M, whereby the presence of this second gene variant causes all but one of the plurality of targeted therapies to have a noted resistance to treatment (see panel 510 of FIG. 5A, where osimertinib is indicated (e.g. through a first indicia) as a recommended therapeutic option for a patient testing positive for both the EGFR p.L858R variant EGFR p.T790M, while afatinib, erlotinib, and gefitinib show (e.g. through a second indicia) resistance when both of those variants are present).

By way of another example, a patient may test positive for the BRAF wild type, MET fusion, NRAS wild type and, as a result of this interaction, be sensitive to capmatinib. According to another example, a patient may test positive for the EGFR exon 19 insertion, the KRAS activating mutation and, as a result of this interaction, be resistant to erlotinib, gefitinib, afitinib, and osimertinib. According to yet another example, a patient may test positive for MTOR p.F1888L and MTOR p.L2230V and, as a result of this interaction, be sensitive to sirolimus.

In some embodiments, the panels may include an indicia (e.g. an integer value) illustrative of the number of gene variants identified for a particular piece of clinical information (see panels 510 and 520 of FIG. 5A where panel 510 includes the indicia “2” indicating the presence of the EGFR p.L858R and EGFR p.T790M gene variants; while panel 520 includes the indicia “3” indicating the presence of the EGFR p.C797S, EGFR p.L858R and EGFR p.T790M gene variants).

In some embodiments, and with reference to FIG. 5B, the panels are populated with an identification of the one or more gene variants, a clinical significance ranking (e.g. a classification rating, a tier rating), and a therapeutic recommendation for a particular disease or condition. In other embodiments, and with reference to FIG. 5C, the panels are populated with an identification of the one or more gene variants, a clinical significance ranking, and a therapeutic recommendation for a particular disease or condition including an identification of those one or more drugs that are either suitable or unsuitable (or even less suitable) for treating the particular disease or condition. In some embodiments, those drugs that are suitable for treating the particular disease or condition are visualized to stand-out from those drugs that are unsuitable or less suitable. In some embodiments, therapy sensitive for the disease of interest is marked with a first indicia; and wherein a therapy resistant for the disease of interest is marked with a second indicia. For example, those drugs that are suitable may be presented within a stand-out box and/or may include some general positive indicia (e.g. a “+” sign, a filled in object). Likewise, those drugs that are unsuitable or less suitable may be presented in a comparatively more abstract manner and many include some general negative indicia (e.g. a lighter text shade as compared with a suitable drug, a “−” sign, an “x”). Other suitable indicia are illustrated in FIGS. 8A and 8B.

In yet other embodiments, and with reference to FIG. 5D, the panel may be divided into a first subsection providing the one or more variant identifications, filtering statistics, ranking, and a brief clinical summary. A second subsection may include, as noted above with regard to FIG. 5C, an identification of those drugs that are suitable, unsuitable, or less suitable for treating a particular disease or condition associated with the identified variants. In some embodiments, the panels may include a cross-reference to another panel, e.g. a hyperlink to another panel. In other embodiments, the panels may provide links to other published information or curated information.

In yet further embodiments, and with reference to FIGS. 5G and 5H, the panels may include gene variant information, classification rankings, statistics, and listings of therapeutic recommendations. In some embodiments, and with reference to FIG. 5H, interacting variants may be shown within separate panels, but where a first panel for a first gene variant includes a first colored indicia and where a second panel for a second gene variant includes a second colored indicia.

In some embodiments, the populated panels are sorted according to a clinical significance or classification ranking (this is believed to further enhance the rapid and efficient review of clinical data and information enabling improved (quicker and more accurate) patient care). In some embodiments, clinical significance of any particular variant or group of variants may be divided into a plurality of tiers. By way of example, Tier I-A may represent approved therapy by a regulatory body (e.g. the FDA) as noted in a professional guideline for a particular variant (or grouping of variants). Tier I-B may represent well-powered studies with a consensus from experts in the field for a particular variant (or grouping of variants). Tier II-C may represent approved therapy by a regulatory body for a different disease or condition (e.g. a different tumor type) or an investigational therapy, with support from multiple published studies with some consensus for a particular variant (or grouping of variants). Tier II-D may represent pre-clinical trials or a few case reports without consensus for a particular variant (or grouping of variants). Tier III may represent variants of unknown clinical significance. Tier IV may represent benign or likely benign variant (or grouping of variants). An “unclassified” tier may represent variants that are yet to be classified or studied.

For example, as illustrated in FIG. 5A, panels 510 and 520, both of which contain panels populated with multiple gene variants, are sorted according to their tier ranking and, in particular, panel 510 having a tier rating of I-A is arranged before panel 520 having a tier rating of II-D. In some embodiments, those panels having multiple gene variants are arranged before those panels having a single gene variant. For example, panels 510 and 520 are arranged before panels 530 and 540 (where panels 530 and 540 are further arranged according to their tier rating in accordance with that noted above).

In some embodiments, the representations or panels are generated and populated with retrieved clinically relevant information only if the retrieved information for a particular gene variant or group of gene variants meets a predetermined minimum tier rating. For example, representations or panels may be rendered for clinically relevant information in tiers I-A, I-B, II-C, and II-D. While panels may not be generated for comparatively less clinically relevant identified information, for example information retrieved and ranked into tiers III and the unclassified tier, that information is still made available to the user of the system. For example, the content renderer 124, after receiving relevant information from content fetcher 122, may generate a list of those Tier III and Unclassified entries, such as depicted in FIG. 5E. For this less clinically relevant information, the content renderer 124 provides at least an identification of the determined gene variants or group of gene variants, the tier rating, certain statistics, and/or the database from which the information was retrieved (see FIG. 5E). In some embodiments, user may interact with those lists of data such that the data may be reclassified into a higher tier (i.e. one that is more clinically relevant) or where the listing may be annotated. In some embodiments, the user may click on any individual listed item and a new window, overlay, or screen will be generated and, as described herein, populated with additional relevant information.

In some embodiments, the GUI renderer 126 displays one or more menu bars having user selectable elements which, when selected, may initiate one or more operations. For example, a menu bar may include a listing of the various tiers such that when the user selects one of the tiers, only panels and other information relevant to those tiers is displayed within the user interface. In some embodiments, the GUI renderer 126 displays an element that, when selected, opens a filtering operations window (described further herein). The filtering operations window may include a plurality of user selectable elements, toggle switches, and boxes for the entry of suitable values (e.g. integer or percentage values) relevant for filtering the retrieved clinically relevant curated information. In yet other embodiments, the GUI renderer 126 displays other user selectable elements, e.g. an element that, when selected, allows for the insertion of text fields, e.g. titles or captions. In even further embodiments, the GUI renderer 126 displays user selectable elements for generating a report (based on the totality of information retrieved for the identified gene variants for the particular disease), approving the report, and/or rejecting the report. In yet even further embodiments, the GUI renderer 126 displays a panel including relevant metadata, such as metadata provided by the system operator or which was included within the variant call format file.

In some embodiments, the information included within any of the one or more populated panels (e.g. either the interacting gene panels or the individual gene panels) may be refined (step 404). In some embodiments, refinement includes filtering data, such as described further herein. For example, a geneticist or other operator may review the clinically relevant information presented within any of the one or more panels and add annotations or modify the information presented therein. By way of further example, the operator may use a user interface device 106 to select certain configuration items which, when selected, remove certain indicated therapeutics from the panel. Finally, a clinical report may be generated based on the curated information presented within the one or more panels (and/or refined by the user) (step 405).

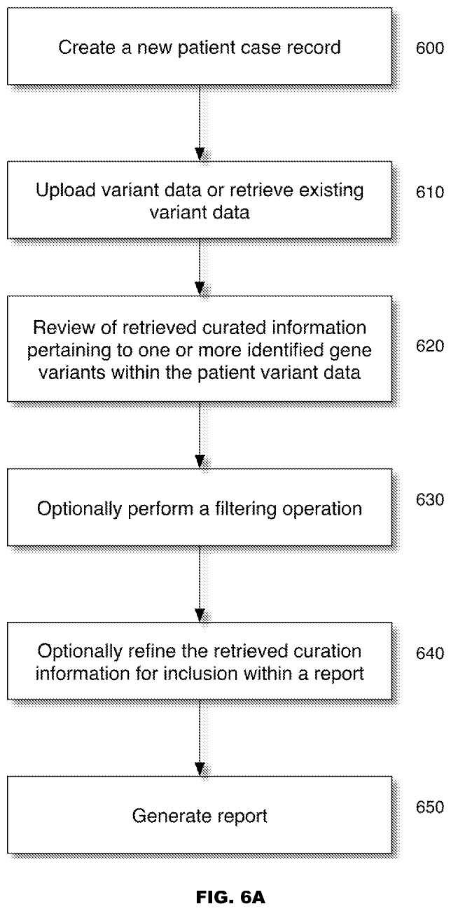

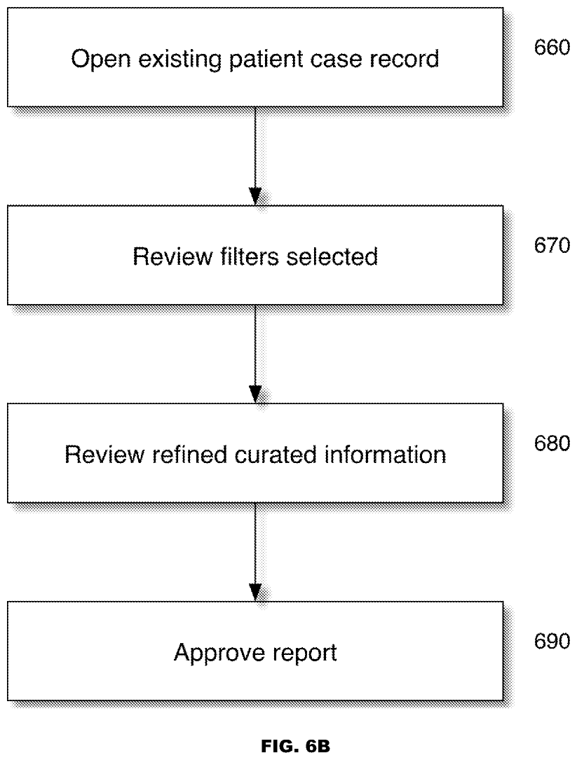

FIG. 6A sets forth a flowchart whereby a remote user may access system 100 to generate a report for a patient based on sequence data obtained from a patient, such as a patient in need of treatment for cancer. In some embodiments, a remote user accesses a client portal and remotely connects to system 100. Once access is attained, a new patient case record may be generated (step 600). Metadata may be added to the new patient case record, including patient name, case identification, patient date of birth, diagnosis, ordering physician, sample collection dates, sample type, etc.). A file containing variant data is then uploaded (step 610) to system 100 and the remote user also indicates the disease or condition in which the patient has been diagnosed. Following the upload of the variant data, the system 100 parses the uploaded data and determines one or more gene variants within the upload information and identifies clinically relevant information based on the one or more determined gene variants (see FIG. 4). Also, as noted above, representations or panels are generated by system 100 such that the clinically relevant information may be presented to the remote user, facilitating review of the identified clinically relevant information (step 620). In some embodiments, the remote user may initiate a filtering operation by interacting with certain interface elements such that user configurable filtering settings may be established (step 630). Following optional filtering, the remote user may then refine the clinically relevant information identified (and/or filtered information) (step 640). For example, the remote user may reclassify certain information and/or provide summaries or other annotations. After refinement, a report may be generated for review (650). In some embodiments, the report generated is a draft report for approval by a second party, e.g. another geneticist, a supervisor. In other embodiments, the report generated is a final report ready for transmission to a clinician (e.g. a treating physician).

FIG. 6B sets forth a flowchart whereby a remote user (e.g. another geneticist, a laboratory supervisor, etc.) may access system 100 to review and/or approve a report which has been previously generated (from step 650). In some embodiments, a remote user again attains remote access to system 100 and subsequently opens or otherwise retrieves an existing patient case record (step 660). The remote user may then review the filters selected (step 670) and the refined curated information (step 680). The remote user then may make a determination as to whether to approve the report or not (step 690). In some embodiments, the remote user will make a determine if the identified and refined curated information is relevant, needs further refinement, and/or is useful for a clinician to make a treatment decision for the patient in need of treatment thereof. In some embodiments, the approved report is transmitted to the clinician. In other embodiments, the approved report is made accessible through a client portal for retrieval, i.e. stored on storage subsystem 104 or a network attached storage device until retrieved.

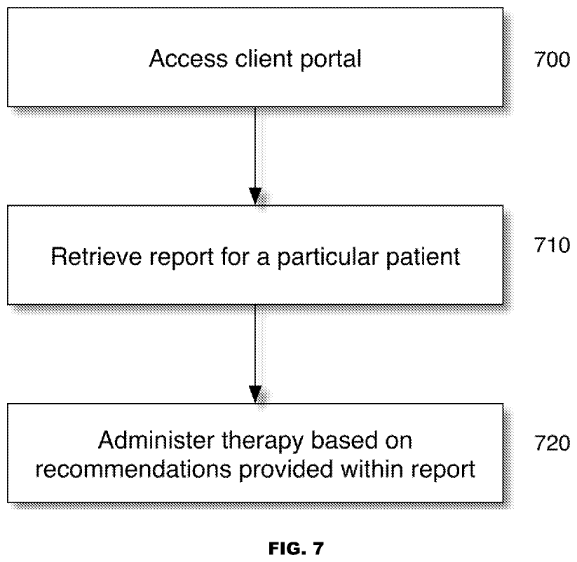

FIG. 7 illustrates a flowchart whereby a remote user, e.g. a clinician, may access system 100 to retrieve an approved report. In some embodiments, the remote user will access system 100 (step 700) to retrieve the approved report (step 710). The remote user would then review the report and make a decision as to a therapeutic approach, e.g. in the context of a patient inflicted with cancer, administer a recommended targeted therapy based on the recommendations provided within the report tying clinical relevant information to the patient's determined one or more gene variants (step 720).

As noted herein, in some embodiments, the gene variants determined from the subject's sequence data may be filtered. In some embodiments, the GUI renderer 126 may provide a pop-up window or drop-down menu including one or more user configurable filtering settings or parameters. In some embodiments, the determined gene variants may be filtered using one or more quality metrics. In some embodiments, quality metrics include a read depth filter and a variant allele frequency filter. In some embodiments, the read depth filter considers the number of the times the variant was detected or measured within the same. In some embodiments, the user may enter an integer value for the read depth filter and the software may filter based on the inputted integer value. In some embodiments, the variant allele frequency filter considers how often the gene variant is present in proportion to the total number of reads. In some embodiments, the user may input a percentage value variant allele frequency filter and the software may filter based on the inputted percentage value.

In some embodiments, determined gene variants may be filtered by evaluating whether the identified clinically relevant information is present or absent in one or more databases, such as databases cataloging information on somatic mutations and/or germline mutations. Depending on the filtering settings and the databases evaluated, data may be included in or excluded from any visualization or report. The goal of those filters is to remove (filter away) a variant if it is not present in a public database, e.g. Catalog of Somatic Mutations in Cancer (“COSMIC”). For example, COSMIC lists variants that are commonly seen in, for example, lung cancer. A user of the variant analysis and reporting software might want to remove variants that have never been observed in lung cancer (as per COSMIC) and only focus on variants that have been seen at least once. This way, the triage process of going through all variants is quicker. Another use case is population frequency filtering (ExAC). A customer might want to remove (filter away) variants that are often seen in the target population, i.e. they could set a filter to exclude variants that are seen above (e.g.) 5% in the population

In some embodiments, user configurable settings may be selected to evaluate whether a particular somatic mutation is “found in” or “confirmed in” one particular database or another. In some embodiments, a user may enter a particular integer value, and the data may be filtered based on whether there are at least the entered integer value quantity of data sets (e.g. samples, cases, etc.) present within the database for the determined gene variants. In some embodiments, the database is the Catalog of Somatic Mutations in Cancer (COSMIC). In other embodiments, the database the Cancer Genome Atlas available from the National Institutes of Health. In some embodiments, the database is the Exome Aggregation Consortium database. By way of example, a filter could include the following parameters: (i) only include if seen in COSMIC at least once (# samples >=1); (ii) only include if Read Depth is more or equal than 500 reads (RD>=500); (iii) only include if Variant Allele frequency is above or equal to 2% (VAF>=2%).

In some embodiments, gene variant data is automatically filtered. In some embodiments, different automatic filters may be applied depending on the type of disease or condition the patient has. In some embodiments, different automatic filters may be applied depending on the assay utilized. For example, the system may be configured to apply a default filter set for all VCFs coming from the same assay.

### Additional Embodiments

In one aspect of the present disclosure is a method of summarizing clinical information relevant to a patient's diagnosed disease (e.g. cancer) based on the presence of one or more gene mutations in a nucleic acid sample derived from the patient, the method comprising: (i) determining a plurality gene variants within sequence data derived from the patient's nucleic acid sample; (ii) matching each of the plurality of determined gene variants to metadata fields within a database of curated clinical information; (iii) identifying clinically relevant information in the curated database pertaining to the patient's diagnosed disease for each of the matches between the determined gene variants and the database metadata fields; and (iv) rendering visualizations (such as within a user interface) incorporating at least some of the identified clinically relevant information for each of the matches, wherein at least one of the visualizations includes data pertaining to an interaction between at least two gene variants of the plurality of determined gene variants. In some embodiments, the method further comprises generating a report including at least some of the rendered visualizations. In some embodiments, the method further comprises transmitting the report to a clinician, such as for the treatment of the patient's diagnosed disease.

In some embodiments, the data pertaining to the interaction of the at least two of the plurality of determined gene variants is a summary of a clinical report or journal article describing the effect of the interaction. In some embodiments, the data pertaining to the interaction of the at least two of the plurality of determined gene variants is a therapeutic recommendation. In some embodiments, therapeutic recommendation is not to administer a particular drug or treatment protocol. In some embodiments, the therapeutic recommendation is a targeted biotherapy approved by a regulatory agency. In some embodiments, the therapeutic recommendation is a treatment protocol approved by a regulatory agency.

In some embodiments, the rendered visualizations include individual gene panel representations and interacting gene panel representations. In some embodiments, the interacting gene panel representations include the data pertaining to the interaction of the at least two identified gene variants of the plurality of determined gene variants. In some embodiments, the interacting gene panel representations include a first portion listing the at least two identified gene variants of the plurality of determined gene variants, and a second portion reporting a therapeutic recommendation. In some embodiments, the therapeutic recommendation is a therapy sensitive for the patient's disease in the presence of the interaction. In some embodiments, the therapeutic recommendation is an off-label use of a therapeutic agent which is otherwise approved for a different disease or condition. In some embodiments, the therapy sensitive for the patient's disease is marked within the second panel with a first indicia (e.g. bold text, a stand-out box, a “+” sign, etc.). In some embodiments, therapy resistant to the patient's disease is marked within the second panel with a second indicia (e.g. comparatively lighter or differently colored text, a “−” sign, an “X”, etc.). In some embodiments, the panels are interactive panels, such that by clicking on the panel, a new window or screen is generated which provides further information or more detailed information as compared within the information provided within the panel.

In some embodiments, the individual gene panel representations incorporate the at least some of the identified clinically relevant information for each of the matches. In some embodiments, individual gene panels include an identification of only a single gene (such as those genes which do not interact, or those genes that do interact). In some embodiments, the individual gene panel representations further include at least one of a variant allele frequency statistic or a read depth statistic. In some embodiments, the individual gene panel representations and the interacting gene panel representations include a classification rating (e.g. a tier rating as described herein).

In some embodiments, the method further comprises filtering the determined plurality of gene variants. In some embodiments, the filtering of the determined plurality of gene variants comprises applying at least one quality metric filter. In some embodiments, the at least one quality metric filter is selected from the group consisting of a read depth filter and a variant allele frequency filter. In some embodiments, the filtering of the determined plurality of gene variants comprises evaluating whether the identified clinically relevant information is present or absent in one or more genetic databases. In some embodiments, the one or more genetic databases are somatic mutation databases and germline mutation databases. In some embodiments, the one or more genetic databases are cured evidence statement databases, population frequency databases, and/or protein outcome prediction databases.

In some embodiments, the method further comprises annotating the displayed visualizations (e.g. modifying information, adding a summary, changing a classification or tier rating, adding or removing therapies, etc.). In some embodiments, patient's disease is a genetic disease. In some embodiments, the patient's disease is an inherited disease. In some embodiments, the patient's disease is a form of cancer. In some embodiments, the cancer is unresponsive to first-line therapy and/or second-line therapy.

In another aspect of the present disclosure is a method of summarizing clinical information curated for a patient in need of treatment for a diagnosed disease (e.g. cancer), the method comprising: (i) receiving variant data derived from a nucleic acid sample obtained from the patient; (ii) receiving, on a graphical user interface, a first user input corresponding to user configurable filtering settings such that the received variant data may be filtered; (iii) displaying, on the graphical user interface, an identification of clinically relevant information pertaining to an interaction of at least two gene variants derived from the filtered variant data, and wherein the identified information pertains to the patient's diagnosed disease; (iv) receiving, on the graphical user interface, a second user input corresponding to user refinements of the identified clinically relevant information pertaining to the interaction of the at least two gene variants; and (v) displaying, on the graphical user interface, a visualization including refined identified clinically relevant information pertaining to the interaction of at least two gene variants. In some embodiments, the method further comprises generating a report including at least some of the visualizations.

In some embodiments, the user configurable filtering settings include user configurable variant quality settings and user configurable genetic database presence settings. In some embodiments, the user configurable variant quality settings include read depth and variant allele frequency. In some embodiments, the user refinements comprise (i) adding annotations; (ii) including or excluding therapeutic options from the identified clinically relevant information; and (iii) reclassifying a tier rating of identified clinically relevant information.

In some embodiments, the visualizations are interacting gene panel representations having a first portion listing the at least two gene variants that are interacting, and a second portion reporting a therapeutic recommendation. In some embodiments, the therapeutic recommendation is a therapy sensitive for the patient's disease in the presence of the gene interaction. In some embodiments, the therapy sensitive for the patient's disease is marked within the second panel with a first indicia. In some embodiments, therapy resistant to the patient's disease is marked within the second panel with a second indicia.

In some embodiments, the method further comprises displaying, on the graphical user interface, the identification of clinically relevant information pertaining to one or more individual gene variants derived from the filtered variant data. In some embodiments, the method further comprises displaying one or more individual gene panel representations. In some embodiments, the individual gene panel representations include a first portion listing the individual gene variant, and a second portion reporting a therapeutic recommendation for the individual gene variant. In some embodiments, the individual gene panel representations further include at least one of a variant allele frequency statistic or a read depth statistic.

In another aspect of the present disclosure is a system for reporting clinically significant information for a patient in need of treatment, the system comprising: (i) one or more processors, and (ii) a memory coupled to the one or more processors, the memory to store computer-executable instructions that, when executed by the one or more processors, cause the system to perform operations comprising: determining a plurality of genetic variants by parsing received variant data derived from a sequenced nucleic acid sample from the patient; retrieving clinically relevant information pertaining each of the determined plurality of genetic variants from a curated database; displaying panels populated with the retrieved clinically relevant information, wherein at least one panel includes an identification of an interaction between at least two gene variants of the determined plurality of genetic variants; and compiling a report including the displayed panels.

In some embodiments, the identification of the interaction between the at least two determined gene variants of the plurality of determined gene variants is a summary of a clinical report describing the effect of the interaction. In some embodiments, the identification of the interaction between the at least two determined gene variants of the plurality of determined gene variants is a therapeutic recommendation. In some embodiments, therapeutic recommendation is not to administer a particular drug or treatment protocol. In some embodiments, the panels include individual gene panel representations and interacting gene panel representations.

In some embodiments, the interacting gene panel representations include the data pertaining to the interaction of the at least two determined gene variants of the plurality of determined gene variants. In some embodiments, the interacting gene panel includes a first portion listing the at least two determined gene variants of the plurality of determined gene variants, and a second portion reporting a therapeutic recommendation. In some embodiments, the therapeutic recommendation is a therapy sensitive for the patient's disease in the presence of the gene interaction. In some embodiments, each individual gene panel representation incorporates the at least some of the retrieved clinically relevant information for one of the determined gene variants. In some embodiments, wherein the individual gene panel representation further includes at least one of a variant allele frequency statistic or a read depth statistic.

In some embodiments, the system further comprises instructions for refining the determined plurality of gene variants or the retrieved clinically relevant information. In some embodiments, the refinement of the determined plurality of gene variants comprises applying at least one quality metric filter. In some embodiments, the at least one quality metric filter is selected from the group consisting of a read depth filter and a variant allele frequency filter. In some embodiments, the refinement of the retrieved clinically relevant information comprises adding one or more annotations.

In another aspect of the present disclosure is a non-transitory computer-readable medium storing instructions for summarizing clinical information relevant to a patient's diagnosed disease based on the presence of one or more gene mutations in a sample derived from the patient, the method comprising: determining a plurality gene variants within sequence data derived from the patient's sample; matching each of the plurality of determined gene variants to metadata fields within a database of curated clinical information; identifying clinically relevant information pertaining to the patient's diagnosed disease for each of the matches between the determined gene variants and the database metadata fields; and displaying visualizations incorporating at least some of the identified clinically relevant information for each of the matches, wherein at least one of the visualizations includes data pertaining to an interaction of at least two determined gene variants of the plurality of determined gene variants.

In some embodiments, instructions are included for generating a report based on the displayed visualizations. In some embodiments, the displayed visualizations include individual gene panel representations and interacting gene panel representations. In some embodiments, the interacting gene panel representations include the data pertaining to the interaction of the at least two determined gene variants of the plurality of determined gene variants. In some embodiments, the interacting gene panel includes a first portion listing the at least two determined gene variants of the plurality of determined gene variants, and a second portion reporting a therapeutic recommendation.

In another aspect of the present disclosure is a method of presenting on a display coupled to a computing device relevant information pertaining to the presence of one or more gene mutations in a sample derived from a patient, the method comprising: accessing one or more databases to identify a clinically relevant interaction between at least two gene variants from an obtained plurality of gene variants within sequence data derived from the patient's sample; accessing; and displaying, on the display, at least one visualization of the identified clinically relevant interaction between the at least two gene variants.

In another aspect of the present disclosure is a method of presenting on a display coupled to a computing device relevant information pertaining to the presence of one or more gene mutations in a sample derived from a patient, the method comprising: obtaining, from a memory coupled to the computing device, a plurality of gene variants within sequence data derived from the patient's sample; accessing one or more databases to identify a clinically relevant interaction between at least two gene variants from the plurality of obtained gene variants; and displaying, on the display, at least one visualization of the identified clinically relevant interaction between the at least two gene variants.

In some embodiments, the at least one visualization of the identified clinically relevant interaction comprises a visual representation of at least one therapeutic recommendation associated with the identified clinically relevant interaction between the at least two gene variants. In some embodiments, the at least one therapeutic recommendation is not to administer a particular drug or treatment protocol that is otherwise recommended for at least one of the at least two gene variants. In some embodiments, the particular drug or treatment protocol is identified but visually distinguished from at least one other drug or treatment protocol recommended for at least one of the at least two gene variants. In some embodiments, the therapeutic recommendation is a therapy sensitive for a disease of interest. In some embodiments, the therapy sensitive for the disease of interest is marked with a first indicia; and wherein a therapy resistant for the disease of interest is marked with a second indicia. In some embodiments, the second indicia is text which is greyed out. In some embodiments, the first indicia is a solid symbol; and wherein the second indicia is a hollow symbol.

In some embodiments, the visual representation is an interacting gene panel representation. In some embodiments, the interacting gene panel representation includes a first portion listing the at least two obtained gene variants for which the clinically relevant interaction was identified, and a second portion reporting the therapeutic recommendation. In some embodiments, the visual representation is an individual gene panel representation. In some embodiments, the individual gene panel representation includes a first portion listing one of the at least two obtained gene variants for which the clinically relevant interaction was identified, and a second portion reporting the therapeutic recommendation.

In some embodiments, the identified clinically relevant interaction pertains to a disease of interest, the method further comprising obtaining the disease of interest from the memory. In some embodiments, the disease of interest is a pre-diagnosed disease. In some embodiments, the pre-diagnosed disease is cancer. In some embodiments, the one or more databases are stored on one or more remote servers. In some embodiments, the one or more databases comprise curated clinical information. In some embodiments, at least one of the one or more databases comprises clinical information pertaining to the interaction of gene variants. In some embodiments, a first database is accessed for retrieval of clinically relevant information pertaining to at least a first obtained gene variant from the plurality of obtained gene variants for a disease of interest; and wherein a second database is accessed for retrieval of clinically relevant information pertaining to an interaction between the at least the first obtained gene variant and at least a second obtained gene variant from the plurality of obtained gene variants for the same disease of interest. In some embodiments, the method further comprises generating a report comprising the at least one visualization. In some embodiments, at least two visualizations of the identified clinically relevant interaction between the at least two gene variants are displayed, wherein one of the at least two visualizations includes an individual gene panel representation. In some embodiments, the method further comprises filtering the obtained plurality of gene variants by applying at least one quality metric filter. In some embodiments, accessing the one or more databases comprises accessing a first and a second table stored in the one or more databases, the first table comprising information about the gene variants, and wherein the second table comprises information about gene variant interactions.

In another aspect of the present disclosure is a computing device comprising a display screen, the computing device configured to: obtain a plurality of gene variants from one or more memories communicatively coupled to the computing device, the plurality of gene variants having been derived from sequence data derived from a patient sample; display on the display screen a first representation comprising data pertaining to an identified clinically relevant interaction between at least two gene variants of the plurality of gene variants; and contemporaneously with displaying the first representation, display on the display screen a second representation comprising clinically relevant information pertaining to a first of the at least two gene variants for which an interaction was identified.

In another aspect of the present disclosure is a computing device comprising a display screen, the computing device configured to: display on the display screen a first representation comprising data pertaining to an identified clinically relevant interaction between at least two gene variants of a plurality of gene variants; and contemporaneously with displaying the first representation, display on the display screen a second representation comprising clinically relevant information pertaining to a first of the at least two gene variants for which an interaction was identified, wherein the plurality of gene variants are derived from sequence data derived from a patient sample, and wherein the plurality of gene variants are obtained from one or more memories communicatively coupled to the computing device (see FIG. 11).

In some embodiments, a third representation is displayed comprising clinically relevant information pertaining to a second of the at least two gene variants for which an interaction was identified. In some embodiments, the data pertaining to an identified clinically relevant interaction is a therapeutic recommendation based on the interaction between the at least two gene variants. In some embodiments, the therapeutic recommendation is not to administer a particular drug or treatment protocol. In some embodiments, the therapeutic recommendation is a therapy sensitive for a disease of interest. In some embodiments, the therapy sensitive for the disease of interest is marked with a first indicia; and wherein a therapy resistant for the disease of interest is marked with a second indicia. In some embodiments, the first and second representations are displayed within a single panel. In some embodiments, the single panel is an individual gene panel representation. In some embodiments, the second representation is visualized in a first portion of the individual gene panel representation and wherein the first representation is visualized in a second portion of the individual gene panel representation. In some embodiments, the first and second representations are displayed within separate panels. In some embodiments, the first representation is displayed within an interacting gene panel, and wherein the second representation is displayed within an individual gene panel. In some embodiments, the individual gene panel further comprises the first representation and identification of the first of the at least two gene variants. In some embodiments, the identified clinically relevant interaction pertains to a disease of interest. In some embodiments, the disease of interest is a pre-diagnosed disease. In some embodiments, the pre-diagnosed disease is cancer.

In another aspect of the present disclosure is a system for visualizing clinically relevant information pertaining to a disease of interest, the system comprising: (i) one or more processors, and (ii) a memory coupled to the one or more processors, the memory to store computer-executable instructions that, when executed by the one or more processors, cause the system to perform operations comprising: obtaining a plurality of genetic variants stored in one or more memories, the variant data derived from a sequenced nucleic acid sample from a patient; accessing one or more curated databases comprising the clinically relevant information to identify a clinically relevant interaction between at least two genetic variants from the plurality of obtained genetic variants; and displaying at least one visualization on a display screen comprising the identified clinically relevant interaction.

In some embodiments, the at least one visualization comprising the identified clinically relevant interaction is a representation providing one or more therapeutic recommendations based on the identified clinically relevant interaction. In some embodiments, the therapeutic recommendation is not to administer a particular drug or treatment protocol. In some embodiments, the therapeutic recommendation is a therapy sensitive for a disease of interest. In some embodiments, the therapy sensitive for the disease of interest is marked with a first indicia; and wherein a therapy resistant for the disease of interest is marked with a second indicia. In some embodiments, the at least one visualization comprises a panel having a first portion listing the at least two determined gene variants for which an interaction was identified, and a second portion reporting a therapeutic recommendation.

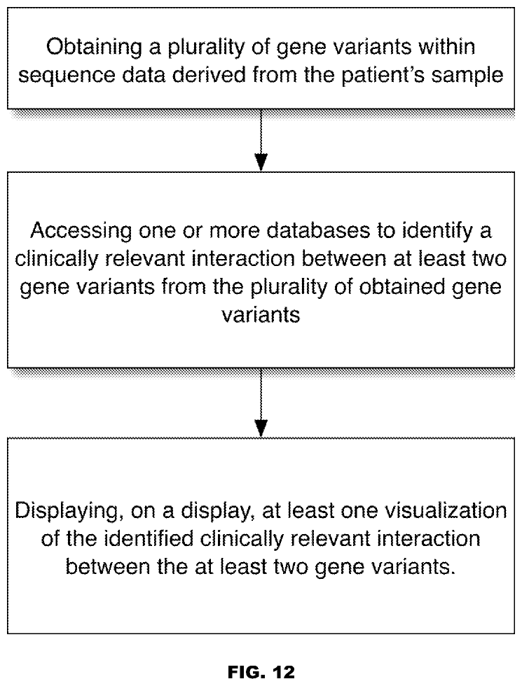

In another aspect of the present disclosure is a method comprising: displaying, on a computing device having a display screen, a first representation comprising data pertaining to an identified clinically relevant interaction between at least two gene variants of a plurality of gene variants; displaying, on the display screen, a second representation comprising clinically relevant information pertaining to at least one of the at least two gene variants for which an interaction was identified, wherein the plurality of gene variants are derived from sequence data derived from a patient sample, and wherein the plurality of gene variants are obtained from one or more memories communicatively coupled to the computing device (see FIG. 12). In some embodiments, the data pertaining to an identified clinically relevant interaction is a therapeutic recommendation based on the interaction between the at least two gene variants. In some embodiments, the therapeutic recommendation is not to administer a particular drug or treatment protocol. In some embodiments, wherein the therapeutic recommendation is a therapy sensitive for a disease of interest. In some embodiments, the therapy sensitive for the disease of interest is marked with a first indicia; and a therapy resistant for the disease of interest is marked with a second indicia. In some embodiments, the first and second representations are displayed within a single panel. In some embodiments, the single panel is an individual gene panel representation. In some embodiments, the second representation is visualized in a first portion of the individual gene panel representation and wherein the first representation is visualized in a second portion of the individual gene panel representation. In some embodiments, the first and second representations are displayed within separate panels.

In another aspect of the present disclosure is a non-transitory computer-readable medium storing instructions which, when executed by one or more processors of a computing system, causes the computing system to display on the display screen a first representation comprising data pertaining to an identified clinically relevant interaction between at least two gene variants of a plurality of gene variants, and to additionally display on the display screen a second representation comprising clinically relevant information pertaining to at least one of the at least two gene variants for which an interaction was identified, wherein the plurality of gene variants are derived from sequence data derived from a patient sample, and wherein the plurality of gene variants are obtained from one or more memories communicatively coupled to the computing device. In some embodiments, the data pertaining to an identified clinically relevant interaction is a therapeutic recommendation based on the interaction between the at least two gene variants.

Embodiments of the subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, data processing apparatus. Any of the modules described herein may include logic that is executed by the processor(s). “Logic,” as used herein, refers to any information having the form of instruction signals and/or data that may be applied to affect the operation of a processor. Software is an example of logic.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, disks, or other storage devices). The operations described in this specification can be implemented as operations performed by a data processing apparatus on data stored on one or more computer-readable storage devices or received from other sources.

The term “programmed processor” encompasses all kinds of apparatus, devices, and machines for processing data, including by way of example a programmable microprocessor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random-access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user and a keyboard and a pointing device, e.g., a mouse or a trackball, by which the user can provide input to the computer. In some implementations, a touch screen can be used to display information and receive input from a user. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks). For example, the network 20 of FIG. 1 can include one or more local area networks.

The computing system can include any number of clients and servers. A client and server are generally remote from each other and typically interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Additional Embodiment 1: A computing device comprising a display screen, the computing device configured to: obtain a plurality of gene variants from one or more memories communicatively coupled to the computing device, the plurality of gene variants having been derived from sequence data derived from a patient sample; and display on the display screen a first representation comprising data pertaining to an identified clinically relevant interaction between at least two gene variants of the plurality of gene variants. In some embodiments, the computing device is further configured to display a second representation (along with the first representation) on the display screen comprising clinically relevant information pertaining to a first of the at least two gene variants for which an interaction was identified.

Additional Embodiment 2: The computing device of additional embodiment 1, wherein a third representation is displayed comprising clinically relevant information pertaining to a second of the at least two gene variants for which an interaction was identified.

Additional Embodiment 3: The computing device of additional embodiment 1, wherein the data pertaining to an identified clinically relevant interaction is a therapeutic recommendation based on the interaction between the at least two gene variants.

Additional Embodiment 4: The computing device of additional embodiment 3, the therapeutic recommendation is not to administer a particular drug or treatment protocol.

Additional Embodiment 5: The computing device of additional embodiment 3, wherein the therapeutic recommendation is a therapy sensitive for a disease of interest.

Additional Embodiment 6: The computing device of additional embodiment 5, wherein the therapy sensitive for the disease of interest is marked with a first indicia; and wherein a therapy resistant for the disease of interest is marked with a second indicia.

Additional Embodiment 7: The computing device of additional embodiment 1, wherein the first and second representations are displayed within a single panel.

Additional Embodiment 8: The computing device of additional embodiment 7, wherein the single panel is an individual gene panel representation.

Additional Embodiment 9: The computing device of additional embodiment 8, wherein the second representation is visualized in a first portion of the individual gene panel representation and wherein the first representation is visualized in a second portion of the individual gene panel representation.

Additional Embodiment 10: The computing device of additional embodiment 1, wherein the first and second representations are displayed within separate panels.

Additional Embodiment 11: The computing device of additional embodiment 10, wherein the first representation is displayed within an interacting gene panel, and wherein the second representation is displayed within an individual gene panel.

Additional Embodiment 12: The computing device of additional embodiment 11, wherein the individual gene panel further comprises the first representation and an identification of the first of the at least two gene variants.

Additional Embodiment 13: A method comprising: displaying, on a computing device having a display screen, a first representation comprising data pertaining to an identified clinically relevant interaction between at least two gene variants of a plurality of gene variants; displaying, on the display screen, a second representation comprising clinically relevant information pertaining to at least one of the at least two gene variants for which an interaction was identified, wherein the plurality of gene variants are derived from sequence data derived from a patient sample, and wherein the plurality of gene variants are obtained from one or more memories communicatively coupled to the computing device.

Additional Embodiment 14: The method of additional embodiment 13, wherein the data pertaining to an identified clinically relevant interaction is a therapeutic recommendation based on the interaction between the at least two gene variants.

Additional Embodiment 15: The method of additional embodiment 14, the therapeutic recommendation is not to administer a particular drug or treatment protocol.

Additional Embodiment 16: The method of additional embodiment 13, wherein the therapeutic recommendation is a therapy sensitive for a disease of interest.

Additional Embodiment 17: The method of additional embodiment 16, wherein the therapy sensitive for the disease of interest is marked with a first indicia; and wherein a therapy resistant for the disease of interest is marked with a second indicia.

Additional Embodiment 18: The method of additional embodiment 13, wherein the first and second representations are displayed within a single panel, wherein the single panel is an individual gene panel representation.

Additional Embodiment 19: The method of additional embodiment 18, wherein the second representation is visualized in a first portion of the individual gene panel representation and wherein the first representation is visualized in a second portion of the individual gene panel representation.

Additional Embodiment 20: The method of additional embodiment 13, wherein the first and second representations are displayed within separate panels.

Additional Embodiment 21: A method of presenting on a display coupled to a computing device relevant information pertaining to the presence of one or more gene mutations in a sample derived from a patient, the method comprising: obtaining, from a memory coupled to the computing device, a plurality of gene variants within sequence data derived from the patient's sample; accessing one or more databases to identify a clinically relevant interaction between at least two gene variants from the plurality of obtained gene variants; and displaying, on the display, at least one visualization of the identified clinically relevant interaction between the at least two gene variants.

Additional Embodiment 22: The method of additional embodiment 21, wherein the at least one visualization of the identified clinically relevant interaction comprises a visual representation of at least one therapeutic recommendation associated with the identified clinically relevant interaction between the at least two gene variants.

Additional Embodiment 23: The method of additional embodiment 22, wherein the at least one therapeutic recommendation is not to administer a particular drug or treatment protocol that is otherwise recommended for at least one of the at least two gene variants.

Additional Embodiment 24: The method of additional embodiment 23, wherein the particular drug or treatment protocol is identified but visually distinguished from at least one other drug or treatment protocol recommended for at least one of the at least two gene variants.

Additional Embodiment 25: The method of additional embodiment 22, wherein the therapeutic recommendation is a therapy sensitive for a disease of interest, and wherein the therapy sensitive for the disease of interest is marked with a first indicia; and wherein a therapy resistant for the disease of interest is marked with text which is greyed out.

Additional Embodiment 26: The method of additional embodiment 22, wherein the visual representation is an interacting gene panel representation, wherein the interacting gene panel representation includes a first portion listing the at least two obtained gene variants for which the clinically relevant interaction was identified, and a second portion reporting the therapeutic recommendation.

Additional Embodiment 27: The method of additional embodiment 22, wherein the visual representation is an individual gene panel representation and wherein the individual gene panel representation includes a first portion listing one of the at least two obtained gene variants for which the clinically relevant interaction was identified, and a second portion reporting the therapeutic recommendation.

Additional Embodiment 28: The method of additional embodiment 22, wherein the one or more databases are stored on one or more remote servers, and wherein the one or more databases comprise curated clinical information, and wherein at least one of the one or more databases comprises clinical information pertaining to the interaction of gene variants.

Additional Embodiment 29: The method of additional embodiment 28, wherein accessing the one or more databases comprises accessing a first and a second table stored in the one or more databases, the first table comprising information about the gene variants, and wherein the second table comprises information about gene variant interactions.

Additional Embodiment 30: A non-transitory computer-readable medium storing instructions which, when executed by one or more processors of a computing system, causes the computing system to display on the display screen a first representation comprising data pertaining to an identified clinically relevant interaction between at least two gene variants of a plurality of gene variants, and to additionally display on the display screen a second representation comprising clinically relevant information pertaining to at least one of the at least two gene variants for which an interaction was identified. In some embodiments, the plurality of gene variants are derived from sequence data derived from a patient sample, and wherein the plurality of gene variants are obtained from one or more memories communicatively coupled to the computing device. In some embodiments, the data pertaining to an identified clinically relevant interaction is a therapeutic recommendation based on the interaction between the at least two gene variants.

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

1. A system comprising:
a computing device configured to:
(i) obtain human gene variant data from one or more memories communicatively coupled to the computing device, wherein the obtained human gene variant data is derived from sequence data collected from a tumor tissue sample of a human patient;
(ii) automatically identify clinically relevant information pertaining to the obtained human gene variant data;
(iii) receive a first user input of a pre-diagnosed type of cancer for the human patient;
(iv) automatically apply one or more filters to the identified clinically relevant information pertaining to the obtained human gene variant data to provide a reduced data set for the pre-diagnosed type of cancer;
(iv) based on the provided reduced data set for the pre-diagnosed type of cancer, visualize on a display screen a first representation comprising (i) a therapeutic recommendation based on a determined clinically relevant interaction between at least two human gene variants of the same gene for the pre-diagnosed type of cancer, and (ii) an identification of the at least two human gene variants for which the clinically relevant interaction was determined; and
(v) contemporaneously with visualizing the first representation, and based on the provided reduced data set for the pre-diagnosed type of cancer, visualize on the display screen a second representation comprising clinically relevant information pertaining to a first of the at least two human gene variants for which the clinically relevant interaction was determined, wherein the clinically relevant information pertaining to the first of the at least two human gene variants for which the clinically relevant interaction was determined is selected from the group consisting of summaries of biological and functional information pertaining to the gene variant, cross references to source material, hyperlinks to source material, and gene variant location.

2. The system of claim 1, further comprising a sequencing device.

3. The system of claim 1, wherein the one or more filters comprise read depth filters and variant allele frequency filters.

4. The system of claim 1, wherein a third representation is displayed comprising clinically relevant information pertaining to a second of the at least two gene variants for which an interaction was identified.

5. The system of claim 1, wherein the therapeutic recommendation is not to administer a particular drug, biotherapeutic, or treatment protocol for the pre-diagnosed type of cancer.

6. The system of claim 1, wherein the therapeutic recommendation is a therapy sensitive for the pre-diagnosed type of cancer.

7. The system of claim 6, wherein the therapy sensitive for the pre-diagnosed type of cancer is marked with a first indicia.

8. The system of claim 1, wherein the first and second representations are displayed within a single panel.

9. The system of claim 8, wherein the single panel is an individual gene panel representation.

10. The system of claim 9, wherein the second representation is visualized in a first portion of the individual gene panel representation and wherein the first representation is visualized in a second portion of the individual gene panel representation.

11. The system of claim 1, wherein the first and second representations are displayed within separate panels.

12. The system of claim 11, wherein the first representation is displayed within an interacting gene panel, and wherein the second representation is displayed within an individual gene panel.

13. The system of claim 12, wherein the individual gene panel further comprises the first representation and an identification of the first of the at least two gene variants.

14. The system of claim 1, wherein the computing device is further configured to automatically remove variant data from the obtained human gene variant data if a variant for the pre-diagnosed type of cancer is not present in a public database.

15. A method of treating a human patient inflicted with a pre-diagnosed type of cancer comprising:
obtaining human gene variant data derived from a human genomic sample collected from the human patient;
identifying a plurality of gene variants within the obtained human gene variant data;
receiving a first user input of the pre-diagnosed type of cancer of the human patient;
receiving a second user input of a selection of one or more quality metric filters;
filtering the obtained human gene variant data based on the selection of the one or more quality metric filters within databases specific to the received pre-diagnosed type of cancer to provide a reduced dataset;
based on the provided reduced dataset, provide a first representation comprising (i) at least first and second therapeutic recommendations based on a determined clinically relevant interaction between at least two different human gene variants of the same human gene, wherein the first therapeutic recommendation comprises one or more therapies sensitive for the pre-diagnosed type of cancer, and wherein the second therapeutic recommendation comprises one or more therapies resistant for the pre-diagnosed type of cancer; and (ii) an identification of the at least two different variants of the same human gene for which the clinically relevant interaction was determined; and
contemporaneously with providing the first representation, and based on the received user input, provide a second representation comprising clinically relevant information pertaining to a first of the at least two different variants of the same human gene for which the clinically relevant interaction was determined, wherein the clinically relevant information is selected from the group consisting of summaries of biological and functional information pertaining to the gene variant, cross references to source material, hyperlinks to source material, and gene variant location; and
administering to the human patient at least one of the therapies sensitive for the pre-diagnosed disease or condition as provided within the first representation, wherein the at least one therapeutic recommendation is a targeted biotherapy sensitive to the pre-diagnosed type of cancer.

16. The method of claim 15, wherein the therapy sensitive for the disease of interest is marked with a first indicia; and wherein a therapy resistant for the disease of interest is marked with a second indicia; wherein the first and second indicia are different.

17. The method of claim 15, wherein the targeted biotherapy is an antibody specific to a protein encoded by a gene corresponding to the at least two different variants for which the clinically relevant interaction was identified, wherein the protein is a receptor, a kinase, an auxiliary factor, or a cell membrane protein.

18. The method of claim 15, wherein the one or more quality metric filters comprise read depth filters and variant allele frequency filters.

19. A system comprising: one or more processors; and one or more memories coupled to the one or more processors, the one or more memories storing a plurality of instructions executable by the one or more processors, the plurality of instructions comprising instructions that when executed by the one or more processors cause the one or more processors to perform operations comprising:
receiving via a client portal module human gene variant data, wherein the human gene variant data is derived from sequence data collected from a tumor tissue sample of a human patient having a pre-diagnosed type of cancer;
using a variant analysis module, automatically identifying clinically relevant information pertaining to the received human gene variant data;
receiving via the client portal module a first user input comprising the pre-diagnosed type of cancer for the human patient;
receiving via the client portal a second user input comprising configuration parameters for read depth filters and variant allele frequency filters;
using the variant analysis module, and based on the received configuration parameters, automatically applying the read depth filters and variant allele frequency filters to the identified clinically relevant information pertaining to the received human gene variant data to provide a reduced dataset specific to the pre-diagnosed type of cancer;
using the variant analysis module, determining within the reduced dataset specific to the pre-diagnosed type of cancer at least one clinically relevant interaction between at least two different variants of the same human gene for the pre-diagnosed type of cancer; and
visualizing via the client portal (a) a first representation comprising (i) a therapeutic recommendation based on the determined clinically relevant interaction between the at least two human gene variants of the same gene, and (ii) an identification of the at least two human gene variants for which the clinically relevant interaction was determined; and (b) a second representation comprising clinically relevant information pertaining to a first of the at least two human gene variants for which the clinically relevant interaction was determined, wherein the clinically relevant information pertaining to a first of the at least two human gene variants for which the clinically relevant interaction was determined is selected from the group consisting of summaries of biological and functional information pertaining to the gene variant, cross references to source material, hyperlinks to source material, and gene variant location.

20. The system of claim 19, further comprising instructions to automatically remove variant data from the received human gene variant data if a variant for the pre-diagnosed type of cancer is not present in a public database.
